(19) United States
(12) Patent Application Publication
(10) Pub. No.: US 2025/0263666 A1
(43) Pub. Date: Aug. 21, 2025

(54) USE OF HUMAN UMBILICAL CORD MESENCHYMAL STEM CELL-DERIVED EXOSOMES (hUC-MSC-ES)

(71) Applicant: BEIJINGANZHEN HOSPITAL,CAPITALMEDICAL UNIVERSITY, Beijing (CN)

(72) Inventors: Hua HE, Beijing (CN); Fusheng HAN, Beijing (CN); Lulu LIU, Beijing (CN); Qian MA, Beijing (CN)

(73) Assignee: BEIJINGANZHEN HOSPITAL,CAPITALMEDICAL UNIVERSITY, Beijing (CN)

(21) Appl. No.: 19/194,137

(22) Filed: Apr. 30, 2025

Related U.S. Application Data
(63) Continuation-in-part of application No. PCT/CN2023/143147, filed on Dec. 29, 2023.

(30) Foreign Application Priority Data
Mar. 10, 2023 (CN) 202310227074.8

(51) Int. Cl. C12N 5/0775; A61K 35/28; A61P 9/10; C07K 14/47; C12N 5/00; C12N 15/86

(52) U.S. Cl. CPC C12N 5/0668; A61K 35/28; A61P 9/10; C07K 14/47; C12N 5/526; C12N 15/86; C12N 2510/00; C12N 2740/15043

(57) ABSTRACT

A method for preparing human umbilical cord mesenchymal stem cell-derived exosomes (hUC-MSC-ES) overexpressing an ischemic myocardium-targeting peptide (IMTP) is provided, including the following steps: inserting a double-stranded fragment of SEQ ID NO: 1 into a lentiviral vector pCDH-CMV-MCS-EF1-GFP-T2A-puro to obtain a recombinant vector, co-transfecting host cells with the recombinant vector and a packaging system to obtain a lentiviral particles, infecting human umbilical cord mesenchymal stem cells (hUC-MSCs) with the lentiviral particles to obtain hUC-MSCs overexpressing the IMTP, preparing conditioned medium of the hUC-MSCs overexpressing the IMTP, and then collecting the hUC-MSC-ES overexpressing the IMTP from the conditioned medium. HUC-MSC-ES prepared by the preparation method and use thereof, as well as a pharmaceutical composition including the hUC-MSC-ES are provided. A preservation solution for the hUC-MSC-ES is also provided.

### CROSS REFERENCE TO RELATED APPLICATION

The present application is a continuation-in-part application of International Patent Application No. PCT/CN2023/143147, filed on Dec. 29, 2023, which claims priority to the Chinese Patent Application No. 202310227074.8, filed with the China National Intellectual Property Administration (CNIPA) on Mar. 10, 2023, and entitled “USE OF HUMAN UMBILICAL CORD MESENCHYMAL STEM CELL-DERIVED EXOSOMES (hUC-MSC-ES)”, which is incorporated herein by reference in its entirety.

### REFERENCE TO SEQUENCE LISTING

A computer readable XML file entitled “GWPCTP20241208215_seqlist”, that was created on Apr. 10, 2025, with a file size of about 7,640 bytes, contains the sequence listing for this application, has been filed with this application, and is hereby incorporated by reference in its entirety.

### TECHNICAL FIELD

The present disclosure belongs to the technical field of biological preparations, and particularly relates to use of human umbilical cord mesenchymal stem cell-derived exosomes (hUC-MSC-ES).

### BACKGROUND

Cardiovascular diseases represent a significant global public health problem, among which ischemic heart disease (IHD) and the heart failure caused by same are the leading causes of death worldwide. In 2015, 8.917 million people died of IHD worldwide, and the number of patients dying from cardiovascular diseases each year is expected to reach 23.3 million by 2030. At present, the prevalence and mortality rates of IHD in China continue to increase. There were 11.39 million IHD patients as of 2021 in China. Although active clinical measures such as medication, interventional therapy, and surgical revascularization have been conducted to achieve reperfusion of infarcted myocardium and save many lives, they also cause further reperfusion injury, making it difficult to reverse the progression of heart failure. Acute myocardial infarction (AMI) causes irreversible extensive cardiomyocyte (CM) loss and subsequent ventricular remodeling, which remain a main cause of chronic heart failure and permanent disability. To fundamentally improve the efficacy of treatment, it is essential to discover novel methods that can effectively reduce damage and repair damaged myocardium.

In the past 20 years, foundational research has shown that stem cell transplantation may reduce infarct size, promote angiogenesis, and significantly improve cardiac function and prognosis 6. However, clinical studies have shown divergent results on the efficacy of stem cell transplantation: some studies reported positive effects, while a few presented neutral or negative results. In addition, stem cell therapy faces inherent challenges such as low retention and survival, as well as poor differentiation rate, which restricts the efficacy. Moreover, the shortcomings and limitations of different stem cells also limit the clinical application of such a therapy. Currently, among various stem cells, mesenchymal stem cells (MSCs) are the most widely used stem cells in clinical practice due to their ease of acquisition, strong proliferation ability, multiple lineage differentiation potential, low immunogenicity, and immunomodulatory effect, making them safe for allogeneic transplantation.

Human umbilical cord mesenchymal stem cells (hUC-MSCs) are more primitive than adult MSCs, showing stronger proliferation and self-renewal abilities. They are also easy to obtain, collect, and preserve. Moreover, hUC-MSCs are derived from medical waste, such that they have no harm or pain to the donors during collection, nor are they subject to ethical, legal, and moral restrictions. Furthermore, hUC-MSCs demonstrate lower tumorigenicity and possibility of viral and bacterial contamination than bone marrow mesenchymal stem cells (BW-MSCs). They are abundant, possess stronger telomerase activity, have short doubling time, and exhibit lower immunogenicity, which leads to minimal immune responses during allogeneic transplantation. Studies have shown that hUC-MSCs have the potential to differentiate into a variety of cells, including osteocytes, adipocytes, cardiomyocytes (CMs), and neuron-like cells, and therefore may be ideal seed cells for tissue repair in cell therapy. Studies in porcine AMI models have shown that hUC-MSCs can improve myocardial perfusion, left ventricular function, and ventricular remodeling⁵³, and intravenous injection of allogeneic hUC-MSCs can reduce infarct size and improve cardiac function⁵⁴. Clinical studies have also shown that hUC-MSCs transplantation can improve cardiac function and reduce cardiac remodeling and post-MI heart failure without increasing the risk of adverse reactions^{55,56} However, challenges remain for hUC-MSCs as a cell therapy strategy, such as minimal retention and survival of transplanted cells in target organs, low differentiation rate, tumorigenic risk, and immunogenicity.

It is known that most cells in the body can secrete extracellular vesicles (EVs), which can be further divided into three types according to their diameters. Exosomes (ES), the smallest EVs, have a diameter of 30 nm to 100 nm and contain proteins, lipids, saccharides, DNAs, mRNAs, miRNAs and other non-coding RNAs. The ES serve as the most important EVs as well as a biological nano-scale carrier. Recent studies have shown that ES secreted by MSCs (namely MSC-ES) can replace their parental cells to exert multiple functions, and may play an important role in mediating survival and alleviating remodeling of post-AMI CMs.

MSCs are among the most promising stem cells for clinical applications. However, whole-cell transplantation of MSCs shows little retention and survival of transplant cells, and there is a risk of tumor formation, which can greatly reduce the efficacy. Recent studies have shown that cell-free MSC-ES can overcome the limitations in whole-cell transplantation of MSCs, while almost fully mimicking the beneficial effects of MSCs as their parental cells, showing equivalent or higher efficacy than the MSCs in treatment. Compared with MSCs, MSC-ES have the advantages of small size and convenient movement, can target specific tissues without immunogenicity, can be repeatedly administered, and are more physiologically stable. In addition, the MSC-ES can be rapidly taken up by cells and affect target cells by transferring bioactive molecules, making it a suitable nanoparticle for delivery. Therefore, MSC-ES offer advantages over whole cell therapy.

Reperfusion and restoration of blood supply after AMI can lead to a decrease in ATP, H⁺ accumulation, calcium overload, and reactive oxygen species (ROS) production in CM. These changes can exacerbate ischemic injury and lead to irreversible damage, a phenomenon known as ischemia/reperfusion (I/R) injury (IRI). With the development of revascularization technologies such as thrombolysis, intervention, and surgical bypass surgery, most AMI patients generally achieve reperfusion therapy through drug therapy, thrombus autolysis, or percutaneous coronary intervention (PCI)/coronary artery bypass grafting (CABG). Therefore, myocardial I/R injury (MIRI) is the main reason for poor efficacy and prognosis in AMI patients, and it poses a significant challenge to optimizing the benefits of reperfusion therapy for ischemic myocardium. MIRI is also related to both high mortality and disability rates. Effective reduction of MIRI may significantly improve prognosis and has important clinical significance. Previous studies have found some drugs that can reduce IRI. Some drugs, such as atrial natriuretic peptide, erythropoietin, exenatide, and mitochondrial protective agent cyclosporine A, have even entered the clinical research stage, but are ultimately discontinued due to lack of clinical benefit. To date, there is no clear and effective drug for alleviating MIRI recommended for use in clinical guidelines, nor cellular/cell-free biological drug for alleviating MIRI recommended in the clinical guidelines.

Currently, human umbilical cord mesenchymal stem cell-derived exosomes (hUC-MSC-ES) have not been used to prevent or treat IRI.

### SUMMARY

Therefore, a first objective of the present disclosure is to provide novel use of human umbilical cord mesenchymal stem cell-derived exosomes (hUC-MSC-ES) in preparation of a drug for preventing, treating, or alleviating myocardial ischemia-reperfusion injury (MIRI) and a related disease thereof.

A second objective of the present disclosure is to provide hUC-MSC-ES overexpressing an ischemic myocardium-targeting peptide (IMTP) that can better prevent, treat, or alleviate MIRI and a related disease thereof.

To this end, the present disclosure provides a method for preparing hUC-MSC-ES overexpressing an IMTP having the sequence of SEQ ID NO: 3, including the following steps: inserting a double-stranded fragment of SEQ ID NO: 1 into a lentiviral vector pCDH-CMV-MCS-EF1-GFP-T2A-puro to obtain a recombinant vector, co-transfecting host cells with the recombinant vector and a packaging system to obtain lentiviral particles, infecting human umbilical cord mesenchymal stem cells (hUC-MSCs) with the lentiviral particles to obtain hUC-MSCs overexpressing the IMTP, preparing a conditioned medium of the hUC-MSCs overexpressing the IMTP and then collecting the hUC-MSC-ES overexpressing the IMTP.

In some embodiments, a process of preparing the conditioned medium includes the following steps: resuspending the hUC-MSCs overexpressing the IMTP subcultured in an exosome-free serum medium to allow culture until a cell confluence reaches 80% to 95%, collecting a supernatant, and then extracting ES from the supernatant.

Optionally, the exsome-free serum medium is a Dulbecco's Modified Eagle Medium (DMEM)/F12 medium containing 10% fetal bovine serum (FBS).

Optionally, the cell confluence reaches 80% to 95% after 4 d to 6 d of culture.

Optionally, the host cells are human embryonic kidney 293T cells. The packaging system includes a recombinant vector, a psPAX2 plasmid, and a pMD plasmid.

The present disclosure further provides a method for preparing hUC-MSC-ES, including the following steps:
resuspending hUC-MSCs subcultured in an exsome-free serum medium to allow culture until a cell confluence reaches 80% to 95%, collecting a supernatant, and then extracting ES from the supernatant.

In some embodiments, a process of extracting the ES includes: centrifuging the supernatant at 2,000 g to 3,000 g (such as for 20 min to 40 min) and 8,000 g to 10,000 g (for example, for 30 min to 60 min) in sequence, filtering an obtained new supernatant, centrifuging an obtained filtrate at 100,000 g (for example, for 60 min to 80 min) to remove a liquid, resuspending a remaining precipitate in a buffer to obtain the hUC-MSC-ES solution or the hUC-MSC-ES solution overexpressing the IMTP, and optionally adding a colloidal solution to obtain the ES.

Optionally, the supernatant is further filtered using a 0.22 μm or 0.45 μm filter membrane.

In some embodiments, the precipitate is centrifuged at 100,000 g (for example, for 60 min to 80 min) after resuspended in the buffer to remove a liquid, and a remaining precipitate is resuspended in a buffer to obtain the hUC-MSC-ES solution or the hUC-MSC-ES solution overexpressing the IMTP.

Optionally, the buffer is phosphate-buffered saline (PBS). The PBS has a formula including: 200 mM Na₂HPO₄; 35 mM KH₂PO₄; 2.74 M NaCl; and 53 mM KCl, pH=7.2-7.6.

In some embodiments, the colloidal solution is prepared by heating a preservation solution including 10% weight by volume (w/v) to 30% (w/v) of a plant-derived recombinant human serum albumin, 20 g/L to 40 g/L of trehalose, 30% to 50% of glycerol, 2% to 4% of ectoine, and 0.01 mol/L to 0.02 mol/L of phosphate-buffered saline (PBS); optionally, the colloidal solution and the hUC-MSC-ES solution or the hUC-MSC-ES solution overexpressing the IMTP are at a volume ratio of 1-1.5:1-1.5; and optionally, the preservation solution has a pH value of 7.0 to 7.5.

The present disclosure further provides hUC-MSC-ES or the hUC-MSC-ES overexpressing the IMTP prepared by the preparation method.

The present disclosure further provides a pharmaceutical composition, including hUC-MSC-ES or the hUC-MSC-ES overexpressing the IMTP prepared by the preparation method and a pharmaceutically acceptable auxiliary material.

Further, the pharmaceutically acceptable auxiliary material includes a conventional auxiliary material such as a filler, a solvent, and a buffer. A dosage form of the pharmaceutical composition is selected from the group consisting of an injection, a solution, a tablet, a granule, and a capsule.

The present disclosure further provides use of the hUC-MSC-ES, the hUC-MSC-ES or the hUC-MSC-ES overexpressing the IMTP prepared by the preparation method, or the pharmaceutical composition in at least one of the following items (i) to (v):
i preparation of a drug for preventing, treating, or alleviating myocardial ischemia-reperfusion injury (MIRI) and a related disease thereof;
ii preparation of a drug for reducing an inflammation and/or reducing cardiomyocyte (CM) apoptosis;
iii preparation of a drug for alleviating calcium overload and/or promoting angiogenesis;
iv preparation of a drug for reducing an oxidative stress damage in CM; and
v preparation of a drug for reducing infarct size, improving cardiac function, or alleviating myocardial fibrosis.

In some embodiments, the related disease is one or more selected from the group consisting of inflammation, arrhythmia, ventricular remodeling, myocardial fibrosis, and post-MI heart failure caused by MIRI.

The present disclosure further provides a preservation solution for hUC-MSC-ES, including 10% (w/v) to 30% (w/v) of a plant-derived recombinant human serum albumin, 20 g/L to 40 g/L of trehalose, 30% (v/v) to 50% (v/v) of glycerol, 2% (w/v) to 4% (w/v) of ectoine, and 0.01 mol/L to 0.02 mol/L of PBS.

In some embodiments, the preservation solution has a pH value of 7.0 to 7.5.

The present disclosure further provides a colloidal solution for preserving hUC-MSC-ES, where the colloidal solution is prepared by heating the preservation solution; and preferably, the heating is conducted at 80° C. to 95° C. for 10 min to 30 min.

The present disclosure further provides a method for preserving hUC-MSC-ES, including mixing the colloidal solution with a solution of the hUC-MSC-ES to allow preservation; optionally, the colloidal solution and the solution of the hUC-MSC-ES are at a volume ratio of 1-1.5:1-1.5; and optionally, the preservation is conducted at not greater than-4° C. (such as −4° C. to −80° C.).

In the present disclosure, the ES can be used within 24 h after preparation (within one day). Alternatively, the ES may be stored in the preservation solution (colloidal solution), and then stored at −20° C. for use within 1 month, or stored at −80° C. for use within 6 months.

The present disclosure further provides a method for preventing, treating, or alleviating MIRI and a related disease thereof, including administering to a subject in need thereof the hUC-MSC-ES, the hUC-MSC-ES or the hUC-MSC-ES overexpressing the IMTP prepared by the preparation method, or the pharmaceutical composition.

In some embodiments, the related disease is one or more selected from the group consisting of an inflammation, arrhythmia, ventricular remodeling, myocardial fibrosis, and post-MI heart failure that are caused by the MIRI.

The present disclosure further provides a method for reducing an inflammation and/or CM apoptosis, including administering hUC-MSC-ES, or hUC-MSC-ES or the hUC-MSC-ES overexpressing the IMTP prepared by the preparation method, or the pharmaceutical composition to a subject in need thereof.

The present disclosure further provides a method for reducing calcium overload and/or promoting angiogenesis, including administering to a subject in need thereof the hUC-MSC-ES, the hUC-MSC-ES or the hUC-MSC-ES overexpressing the IMTP prepared by the preparation method, or the pharmaceutical composition.

The present disclosure further provides a method for reducing an oxidative stress damage of CM, including administering to a subject in need thereof the hUC-MSC-ES, the hUC-MSC-ES or the hUC-MSC-ES overexpressing the IMTP prepared by the preparation method, or the pharmaceutical composition.

The present disclosure further provides a method for reducing an infarct size, improving a cardiac function, and alleviating myocardial fibrosis, including administering to a subject in need thereof the hUC-MSC-ES, the hUC-MSC-ES or the hUC-MSC-ES overexpressing the IMTP prepared by the preparation method, or the pharmaceutical composition.

In some embodiments, a route of administration includes but is not limited to oral administration and injection.

In some embodiments, the route of administration is injection.

In some embodiments, the hUC-MSC-ES is administered at 26 μg/kg to 260 μg/kg.

Compared with the prior art, embodiments of the present disclosure have the following advantages:

1. The present disclosure provides a method for preparing hUC-MSC-ES overexpressing an IMTP, including the following steps: inserting a double-stranded fragment of SEQ ID NO: 1 into a lentiviral vector pCDH-CMV-MCS-EF1-GFP-T2A-puro to obtain a recombinant vector, transfecting host cells with the recombinant vector to obtain hUC-MSCs overexpressing the IMTP, preparing a conditioned medium of the hUC-MSCs overexpressing the IMTP, and optionally adding a pharmaceutically acceptable auxiliary material to obtain the hUC-MSC-ES overexpressing the IMTP. The coding DNA sequence (CDS) region of human LAMP2B is fused with the IMTP, and glycine linkers are added in 5′ and 3′ ends of a resulting fused sequence, where GCTCGA is added in the 5′ end, and TCCGGAGGT is added in the 3′ end. A Kozak fragment GCCACC is added in front of the fused sequence to improve the translation efficiency. The restriction sites EcoR I (GAATTC) and BamH I (GGATCC) are added at both ends of the fused sequence. An IMTP+LAMP2B sequence or Blank+LAMP2B sequence (control) of the targeting peptide is integrated into the pCDH-CMV-MCS-EF1-GFP-T2A-puro vector to obtain a recombinant vector. The recombinant vector and a packaging system are co-transfected into host cells to obtain lentiviral particles, and then hUC-MSCs are infected with the lentiviral particles to obtain the hUC-MSC-ES overexpressing the IMTP. Compared with naive hUC-MSC-ES, the hUC-MSC-ES of the present disclosure exhibit a more pronounced effect in preventing, treating, or alleviating MIRI.

2. In the method for preparing the hUC-MSC-ES provided in the present disclosure, a large number of vesicles with varying sizes are released during cell apoptosis/death, and may contaminate the ES produced by viable cells during the purification of the ES. Therefore, it is important to ensure that the proportion of dead cells is less than 5% when the hUC-MSCs are harvested. The subcultured hUC-MSCs are suspended in an exsome-free serum medium to allow culture until a cell confluence reaches 80% to 95%, a supernatant is collected, and then the ES is extracted from the supernatant. If the medium replacement is neglected, the extracted ES may be contaminated by various ES types, affecting the therapeutic effect of the hUC-MSC-ES.

3. The present disclosure provides a preservation solution for hUC-MSC-ES, including 10% (w/v) to 30% (w/v) of a plant-derived recombinant human serum albumin, 20 g/L to 40 g/L of trehalose, 30% to 50% of glycerol, 2% to 4% of ectoine, and 0.01 mol/L to 0.02 mol/L of PBS. The preservation solution is heated to obtain a colloidal solution. The ectoine can balance the osmotic pressure inside and outside the ES membrane, providing protection for enzymes, DNA, cell membranes, and the entire ES from high-temperature, freezing, and drying, and avoiding the possible toxicity of dimethyl sulfoxide (DMSO). It is more efficient and safer. The human serum albumin increases the protein concentration in the ES storage system to prevent proteases from hydrolyzing ES membrane proteins. The trehalose can form a protective film on the surface of ES at low temperatures to protect protein molecules from denaturation and inactivation, thereby maintaining the biological activity of ES. The glycerol protects the preservation solution from freezing at −20° C., preventing the ES from structural damage due to freez-thawing cycles during storage and use. The PBS maintains the osmotic pressure of ES and controls the acid-base balance, protecting the integrity of ES structure and the stability of the contents. The preservation solution can greatly improve the stability of hUC-MSC-ES, such that the hUC-MSC-ES can be stably stored for 1 month at −20° C. and for 6 months at −80° C. without freezing and thawing.

4. The present disclosure provides use of the hUC-MSC-ES, the hUC-MSC-ES overexpressing the IMTP, or the pharmaceutical composition. The effectiveness of hUC-MSC-ES in in vitro CM models and in vivo myocardial I/R models in mammals has been verified from multiple levels. In addition, the long-term safety and therapeutic effect of the treatment are verified by a variety of advanced and reliable technical detection approaches, and possible molecular mechanisms of action are also analyzed. Moreover, the dynamic biological distribution curve and characteristics of the hUC-MSC-ES as a cell derivative in vivo are also analyzed, providing important evidence support for clinical applicability, and exhibiting fully innovative. The hUC-MSC-ES are a biological drug derived from the organism, making them safer.

The hUC-MSCs are derived from medical waste, such that they have no harm or pain to the donors during collection, nor are they subject to ethical, legal, and moral restrictions. Furthermore, the hUC-MSC-ES require relatively simple separation and are abundant. Under constant extraction conditions, the content of ES is relatively stable, which offers advantages such as wide sources, simple separation, high content, and good stability.

The scheme of the present disclosure involves a cell-free therapy. Compared with cell therapy, the hUC-MSC-ES can overcome the limitations of high storage and transportation requirements, low survival and differentiation, and tumor formation risk during whole cell transplantation. The hUC-MSC-ES have a small size and convenient movement, can target specific tissues, shows no immunogenicity, can be repeatedly administered, and are more physiologically stable. The hUC-MSC-ES can be rapidly taken up through cells to affect target cells by transferring bioactive molecules. They are nanoparticles suitable for delivery. Meanwhile, the hUC-MSC-ES can effectively simulate the beneficial effects of their parental cells, hUC-MSCs, and can be equivalent to or more efficient than hUC-MSCs therapy.

Due to their complex components, the hUC-MSC-ES may reduce MIRI and improve the prognosis of AMI through multiple pathways such as reducing apoptosis, alleviating myocardial fibrosis, promoting angiogenesis, and alleviating inflammation through various molecular substances, showing higher efficiency.

### BRIEF DESCRIPTION OF THE DRAWINGS

To describe the technical solutions in the embodiments of the present disclosure or the prior art more clearly, the drawings required are briefly described below. Apparently, the drawings in the following description show merely some embodiments of the present disclosure, and those of ordinary skill in the art may still derive other drawings from these drawings without creative efforts.

FIGS. 1A-1D show the culture and identification results of hUC-MSCs, where FIG. 1A shows the appearance of hUC-MSCs observed under an inverted phase contrast microscope,

FIG. 1B shows the growth curve of hUC-MSCs, FIG. 1C shows the detection result of hUC-MSCs by flow cytometry; FIG. 1D shows the osteogenic induction and adipogenic induction of hUC-MSCs, induced differentiation refers to an experimental group in which adipogenic/osteogenic induction solution was added, and Control refers to a control group in which conventional medium was added;

FIGS. 2A-2F illustrate the construction and identification of IMTP-ES, where FIG. 2A is a schematic diagram showing LAMP2b protein fused to IMTP sequence; FIG. 2B is a photo of fluorescent fibers of hUC-MSCs infected with lentivirus; FIG. 2C shows the expression levels of Lamp2b and IMTP in three groups of hUC-MSCs as detected by RT-PCR; FIG. 2D shows the expression levels of Lamp2b and IMTP in three groups of hUC-MSCs as detected by Western blot; FIG. 2E shows the expression levels of Lamp2b and IMTP in three groups of ES detected by RT-PCR; FIG. 2F shows the expression levels of Lamp2b and IMTP in three groups of ES detected by Western blot; Control-MSCs: naive hUC-MSCs; Blank-MSCs: hUC-MSCs infected with empty vector lentivirus; IMTP-MSCs: hUC-MSCs infected with lentivirus carrying IMTP (also called hUC-MSCs overexpressing IMTP); Control-ES: ES secreted by the naive hUC-MSCs; Blank-ES: ES secreted by the hUC-MSCs infected with empty vector lentivirus; IMTP-ES: ES secreted by the hUC-MSCs infected with lentivirus carrying IMTP (also called hUC-MSC-ES overexpressing IMTP);

FIGS. 3A-3C show the identification results of Control-ES, Blank-ES, and IMTP-ES, where FIG. 3A: morphological characteristics of 3 types of hUC-MSC-ES, Control-ES, Blank-ES, and IMTP-ES shown by transmission electron microscopy (TEM); FIG. 3B: particle size distribution of Control-ES, Blank-ES, and IMTP-ES measured based on nanoparticle tracking analysis; FIG. 3C: the expression levels of CD9 and CD63 in the 3 types of hUC-MSCs-ES, Control-ES, Blank-ES, and IMTP-ES detected by Western blot, with naive hUC-MSCs as control;

FIG. 4 shows the dynamic biological distribution of three types of hUC-MSC-ES (Control-ES, Blank-ES, and IMTP-ES) in normal rats and MIRI rat models, as detected by fluorescence imaging; Control-ES: normal rats are administered Control-ES; I/R+Control-ES: I/R rats are administered Control-ES; I/R+Blank-ES: I/R rats are administered Blank-ES; I/R+IMTP-ES: I/R rats are administered IMTP-ES;

FIG. 5 shows the biological distribution characteristics of three types of hUC-MSC-ES (Control-ES, Blank-ES, and IMTP-ES) in different organs of normal rats and MIRI rat models as detected by fluorescence imaging;

FIG. 6 shows the dynamic distribution of three types of hUC-MSC-ES (Control-ES, Blank-ES, and IMTP-ES) in different parts of the heart tissue in each group of rats as displayed by immunofluorescence, where Normal: non-infarction normal zone; Infarction Border Zone: infarction border zone;

FIG. 7 shows the fluorescence intensity of three types of hUC-MSC-ES (Control-ES, Blank-ES, and IMTP-ES) in different organs of rats in each group as analyzed by immunofluorescence; BZ: MI infarction border zone; RZ: MI non-infarction normal zone; *: P<0.05 in BZ vs. RZ, **: P<0.01 in BZ vs. RZ, NS: no statistical significance;

FIGS. 8A-8H show the changes in various indicators of human embryonic stem cell-derived cardiomyocytes (hESC-CMs) after being treated with different types of hUC-MSC-ES, where FIG. 8A shows the cell viability detected by CCK-8 method; FIG. 8B shows the T-SOD activity detected by chemical method; FIG. 8C shows the MDA level detected by chemical method; FIGS. 8D-8F show the expression levels of TNF-α, IL-1β, and IL-10 in the cell supernatant of each group detected by ELISA; FIG. 8G to FIG. 8H show the expression levels of Bax, cleaved-caspase3, and FasL detected by Western blot and their statistical analysis; Control group: cells routinely cultured without any treatment; H/R group: cells underwent hypoxia/re-glucose/reoxygenation treatment only; H/R+PBS: cells treated with PBS after hypoxia/re-glucose/reoxygenation treatment; H/R+ES: after hypoxia/re-glucose/reoxygenation treatment, three different types of hUC-MSC-ES were added: Control-ES, Blank-ES, and IMTP-ES;

FIGS. 9A-9C show the Ca²⁺ levels in hESC-CMs after different hUC-MSC-ES treatments detected by Fluo-3/AM fluorescent probe and flow cytometry, where FIG. 9A shows the distribution of free Ca²⁺ in each group of cells observed under a fluorescent microscope; FIG. 9B shows the analysis of Ca²⁺ fluorescence intensity in each group of cells; FIG. 9C shows the intracellular Ca²⁺ concentration detected by flow cytometry. ** is P<0.01 vs. Control group; ##is P<0.01 vs. H/R+PBS group; †† is P<0.01 vs. H/R+Control-ES group;

FIG. 10 shows the statistical analysis of the number of tubule branch nodes, cumulative tubule length, and number of tubule Loops formed in each group in the tubule formation assay;

FIG. 11 shows cell apoptosis in cardiac tissues of rats in each group detected by TUNEL staining; Sham group: sham operation group; I/R group: only I/R model without other treatment; I/R+PBS group: I/R model injected with PBS; I/R group+ES: I/R model injected with three different types of hUC-MSC-ES (Control-ES, Blank-ES, IMTP-ES). *: vs. I/R+PBS group, P<0.05; **: vs. I/R+PBS group, P<0.01;

FIGS. 12A-12D show the expression levels of MCP-1, IL-1β, and TNF-α in the myocardium of each group of rats in the I/R model after 24 h of reperfusion, as detected by Western blot, where FIG. 12A shows the expression levels of TNF-α, IL-1β, and MCP-1 in rat myocardial tissue; FIG. 12B shows the quantitative analysis of the expression level of TNF-α, IL-1β, and MCP-1 (**: P<0.01 vs. Sham group; ##: P<0.01 vs. IR+PBS group; **: P<0.01 vs. I/R+Control-ES group); Sham group: sham operation group; I/R group: only I/R model is established without other treatment; I/R+PBS group: I/R model is established and then PBS is injected; I/R group+ES: I/R model is established and then three different types of hUC-MSC-ES are injected (Control-ES, Blank-ES, IMTP-ES);

FIGS. 13A-13D show the oxidative stress level and inflammatory cell infiltration in the myocardial tissue of the infarction border zone in each group of rats after 24 h of reperfusion, where FIG. 13A shows the ROS level in the myocardium of each group of rats observed by DHE fluorescent probe; FIG. 13B shows the statistical analysis of the ROS fluorescence intensity of each group; FIG. 13C is the MDA content in the myocardium of each group of rats; FIG. 13D is HE staining showing the inflammatory cell infiltration of the myocardial tissue in the infarction border zone in each group of rats; *: P<0.05 vs. Sham group; **: P<0.01 vs. Sham group; ##: P<0.01 vs. IR+PBS group; ††: P<0.01 vs. I/R+Control-ES group);

FIG. 14 illustrates the Masson staining analysis of the myocardial tissue in the infarction border zone in each group of rats 3 months after surgery;

FIG. 15 shows the left ventricular end-systolic diameter (LVESD) and ejection fraction (EF) in each group of rats at 4 weeks and 3 months after surgery measured by echocardiography. ***: vs. Sham, P<0.001; ###: vs. I/R+PBS, P<0.001; +++: vs. I/R+Control-ES, P<0.001; A to F represent Sham group (sham operation group), I/R model, I/R+PBS group, I/R+Control-ES, I/R+Blank-ES, and I/R+IMTP-ES group, respectively;

FIGS. 16A-16B show the expression of α-SMA in the myocardial tissue of the infarction border zone of rats in each group at 3 months after surgery as analyzed by immunofluorescence; where FIG. 16A shows the changes in myocardial arteriole formation in different treatment groups (400×); FIG. 16B shows the statistical analysis of myocardial arteriole density in each group; the results show that the myocardial arteriole density increases after treatment with Control-ES and Blank-ES compared with that of the control group; the myocardial arteriole formation in the IMTP-ES treatment group is further increased compared with that in the control-ES and blank-ES groups (blue represents DAPI nuclei, red represents α-SMA);

*: P<0.05 vs. Sham group; **: P<0.01 vs. Sham group; #: P<0.05 vs. I/R+PBS group; ##: P<0.01 vs. I/R+PBS group; †: P<0.05 vs. I/R+Control-ES group;

FIG. 17 shows the functional enrichment changes in hUC-MSC-ES analyzed by whole transcriptome microarray, illustrating the biological process, cellular component, and molecular function significantly upregulated in hUC-MSC-ES compared with those in hUC-MSCs (TOP 10);

FIGS. 18A-18B show the changes in signaling pathway enrichment in hUC-MSC-ES analyzed by transcriptome microarray, illustrating the signaling pathways that are significantly upregulated and downregulated in hUC-MSC-ES compared to hUC-MSCs (TOP 15);

FIG. 19 shows the changes in the signaling pathway interaction network in hUC-MSC-ES analyzed by transcriptome microarray, indicating that apoptosis-related pathways in hUC-MSC-ES are significantly weakened; (P<0.05 vs. hUC-MSCs); 1 presents adherens junction, 2 presents pathways in cancer, 3 presents prostate cancer, 4 presents p53 signaling pathway, 5 presents colorectal cancer, 6 presents chronic myeloid leukemia, 7 presents pancreatic cancer, 8 presents acute myeloid leukemia, 9 presents hepatitis B, 10 presents pathogenic Escherichia coli infection, 11 presents cell cycle, 12 presents proteoglycans in cancer, 13 presents PI3K-Akt signaling pathway, 14 presents regulation of actin cytoskeleton, 15 presents apoptosis, 16 presents shigellosis, 17 presents viral carcinogenesis;

FIG. 20 shows the MIRI rat model after hUC-MSC-ES treatment analyzed by bulk transcriptome sequencing, illustrating the signaling pathways that are significantly upregulated in the myocardial tissue of the infarction border zone (TOP 20);

FIG. 21 is a total t-distributed Stochastic Neighbor Embedding (tSNE) cell projection plot of myocardial tissue as analyzed by 10× Genomics single-cell transcriptome sequencing; where A is Sham group: sham operation group (n=3); B is I/R+PBS group: I/R model injected with 200 μL PBS (n=6); C is I/R+hUC-MSC-ES group: I/R model injected with 400 μg/200 μL hUC-MSC-ES through the tail vein (n=6); B cell_Cd79a high: B cells with high expression of CD79a; CM: cardiomyocyte; SMC: smooth muscle cell;

FIG. 22 shows the changes in proportions of different cellular components in each group of myocardial tissue as analyzed by single-cell transcriptome sequencing; where A is Sham group: sham operation group (n=3); B is I/R+PBS group: I/R model injected with 200 μL PBS (n=6); C is I/R+hUC-MSC-ES group: I/R model injected with 400 μg/200 μL hUC-MSC-ES through the tail vein (n=6);

B cell_Cd79a high: B cells with high expression of CD79a; CM: cardiomyocyte; SMC: smooth muscle cell;

FIGS. 23A-23C show the biological process, cellular component, and molecular function significantly enhanced in CMs of I/R rat model after hUC-MSC-ES treatment (TOP 20), as analyzed by single-cell transcriptome analysis.

### DETAILED DESCRIPTION OF THE EMBODIMENTS

The embodiments of the present disclosure will be described below through specific examples. Unless otherwise specified, the experimental methods disclosed in the present disclosure are all conventional techniques in the art. The reagents and raw materials used in the examples are all commercially available.

### Example 1

This example provides a method for preparing hUC-MSCs overexpressing IMTP, including the following steps:

(1) Isolation of hUC-MSCs

After acquisition of informed consent, the umbilical cord of a full-term cesarean section fetus was collected. The umbilical cord was rinsed with PBS containing 1% penicillin-streptomycin, and its adventitia, umbilical artery and vein were removed to obtain Wharton's jelly tissue. The tissue was rinsed and cut into pieces, placed in Hank's balanced salt solution (HBSS) containing 1 mg/mL type I collagenase, 65 μg/mL DNaseI (deoxyribonuclease I), and 1× penicillin-streptomycin, and digested at 37° C. for 3 h with shaking, filtered through a 70 μm filter, and a collected suspension was centrifuged for 30 min. The centrifuged product was resuspended in DMEM/F12 complete medium containing 10% FBS and then cultured in an incubator at 37° C. with 5% CO₂ for 72 h, where the medium was changed every 3 d. When the cell confluence reached 80%, trypsin was added for digestion and passage to obtain naive hUC-MSCs.

### (2) Construction of Vector Overexpressing IMTP and Lentiviral Packaging

1. The CDS region of the LAMP2B gene (NM_013995.2, CDS: 1233 bp) was derived from Human. The IMTP sequence CSTSMLKAC (IMTP, SEQ ID NO: 4) was inserted behind the LAMP2B signal peptide (the signal peptide was 28 aa long, namely in back of 84th base), corresponding to the base sequence TGTAGCACTTCAATGCTGAAAGCATGT (SEQ ID NO: 3), and glycine linkers were added in front of and behind the sequence, where GCTCGA was added at the 5′ end, and TCCGGAGGT was added at the 3′ end. Finally, a Kozak sequence GCCACC was added in front the sequence to improve the translation efficiency, and an IMTP+LAMP2B insertion sequence was obtained, as shown in SEQ ID NO: 1. A Blank+LAMP2B insertion sequence of the control lentiviral expression vector was obtained by adding the kozac fragment only in front of the CDS region of LAMP2B, as shown in SEQ ID NO: 2.

According to the multiple cloning site of the lentiviral expression vector pCDH-CMV-MCS-EF1-GFP+Puro (purchased from FenghBio, catalog number BR318), the restriction sites Nhe I and BamH I were selected, and the sequence was inserted into the lentiviral expression vector to obtain plasmids pCDH-CMV-[IMTP+LAMP2B]-MCS-EF1-GFP+puro and pCDH-CMV-[Blank+LAMP2B]-MCS-EF1-GFP+puro. The synthesis of the two sequences and the construction of the corresponding lentiviral expression vectors were commissioned to General Biol (Anhui) Co., Ltd.

SEQ ID NO: 1:
GCCACCatggtgtgcttccgcctcttcccggttccgggctcagggctcgt
tctggtctgcctagtcctgggagctgtgcggtcttatgcaGCTCGATGTA
GCACTTCAATGCTGAAAGCATGTTCCGGAGGTttggaacttaatttgaca
gattcagaaaatgccacttgcctttatgcaaaatggcagatgaatttcac
agtacgctatgaaactacaaataaaacttataaaactgtaaccatttcag
accatggcactgtgacatataatggaagcatttgtggggatgatcagaat
ggtcccaaaatagcagtgcagttcggacctggcttttcctggattgcgaa
ttttaccaaggcagcatctacttattcaattgacagcgtctcattttcct
acaacactggtgataacacaacatttcctgatgctgaagataaaggaatt
cttactgttgatgaacttttggccatcagaattccattgaatgacctttt
tagatgcaatagtttatcaactttggaaaagaatgatgttgtccaacact
actgggatgttcttgtacaagcttttgtccaaaatggcacagtgagcaca
aatgagttcctgtgtgataaagacaaaacttcaacagtggcacccaccat
acacaccactgtgccatctcctactacaacacctactccaaaggaaaaac
cagaagctggaacctattcagttaataatggcaatgatacttgtctgctg
gctaccatggggctgcagctgaacatcactcaggataaggttgcttcagt
tattaacatcaaccccaatacaactcactccacaggcagctgccgttctc
acactgctctacttagactcaatagcagcaccattaagtatctagacttt
gtctttgctgtgaaaaatgaaaaccgattttatctgaaggaagtgaacat
cagcatgtatttggttaatggctccgttttcagcattgcaaataacaatc
tcagctactgggatgcccccctgggaagttcttatatgtgcaacaaagag
cagactgtttcagtgtctggagcatttcagataaatacctttgatctaag
ggttcagcctttcaatgtgacacaaggaaagtattctacagcccaagagt
gttcgctggatgatgacaccattctaatcccaattatagttggtgctggt
ctttcaggcttgattatcgttatagtgattgcttacgtaattggcagaag
aaaaagttatgctggatatcagactctgtaa
SEQ ID NO: 2:
GCCACCatggtgtgcttccgcctcttcccggttccgggctcagggctcgt
tctggtctgcctagtcctgggagctgtgcggtcttatgcattggaactta
atttgacagattcagaaaatgccacttgcctttatgcaaaatggcagatg
aatttcacagtacgctatgaaactacaaataaaacttataaaactgtaac
catttcagaccatggcactgtgacatataatggaagcatttgtggggatg
atcagaatggtcccaaaatagcagtgcagttcggacctggcttttcctgg
attgcgaattttaccaaggcagcatctacttattcaattgacagcgtctc
attttcctacaacactggtgataacacaacatttcctgatgctgaagata
aaggaattcttactgttgatgaacttttggccatcagaattccattgaat
gacctttttagatgcaatagtttatcaactttggaaaagaatgatgttgt
ccaacactactgggatgttcttgtacaagcttttgtccaaaatggcacag
tgagcacaaatgagttcctgtgtgataaagacaaaacttcaacagtggca
cccaccatacacaccactgtgccatctcctactacaacacctactccaaa
ggaaaaaccagaagctggaacctattcagttaataatggcaatgatactt
gtctgctggctaccatggggctgcagctgaacatcactcaggataaggtt
gcttcagttattaacatcaaccccaatacaactcactccacaggcagctg
ccgttctcacactgctctacttagactcaatagcagcaccattaagtatc
tagactttgtctttgctgtgaaaaatgaaaaccgattttatctgaaggaa
gtgaacatcagcatgtatttggttaatggctccgttttcagcattgcaaa
taacaatctcagctactgggatgcccccctgggaagttcttatatgtgca
acaaagagcagactgtttcagtgtctggagcatttcagataaataccttt
gatctaagggttcagcctttcaatgtgacacaaggaaagtattctacagc
ccaagagtgttcgctggatgatgacaccattctaatcccaattatagttg
gtgctggtctttcaggcttgattatcgttatagtgattgcttacgtaatt
ggcagaagaaaaagttatgctggatatcagactctgtaa.

(3) Packaging and Lentiviral Infection of hUC-MSCs

i) Packaging: 8 mL of fresh complete medium was added into a 1.50 mL centrifuge tube, which was then pre-warmed, wipe-dried, and placed in a biosafety cabinet. The frozen 293T cells (Zhongqiao Xinzhou, Shanghai) taken out of the liquid nitrogen tank were quickly placed in a 37° C. water bath and shaken rapidly to ensure complete dissolution of the cell suspension within 1-2 min. The cell suspension was quickly added into 8 mL of the pre-warmed fresh complete medium and centrifuged at 1,500 rpm for 5 min. The supernatant was discarded, 5 mL of fresh complete medium was added to resuspend the cell pellet, and then added into a T25 culture flask. The cell pellet was cultured in an incubator at 37° C., 5% CO₂, and 95% relative humidity. The appearance and confluence of cells were observed regularly. When the cell confluence reached 90%, the cells were subcultured at a ratio of 1:3 to expand the cell number to the desired quantity. One day before virus packaging, the cells were trypsinized and inoculated at 1×10⁶ cells/dish in 10 cm culture dishes (NEST). Cell transfection was conducted when the cell confluence was about 70%. When cells were transfected, a shuttle plasmid pCDH-CMV-[IMTP+LAMP2B]-MCS-EF1-GFP+puro (control: pCDH-CMV-[Blank+LAMP2B]-MCS-EF1-GFP+puro) was co-transfected with the packaging plasmids psPAX2 and pMD2.0G. Next, 4 μg of shuttle plasmid, 4 μg of psPAX2 plasmid, and 4 μg of pMD2.0G plasmid were used. During transfection, a mixture of the above three plasmids and 10 μL of P3000 were added into 200 μL of DMEM serum-free medium, while 12 μL of Lipofectamine 3000 reagent (invitrogen, Cat. No.: L3000015) was added into 200 μL of DMEM serum-free medium in another microcentrifuge tube. The diluted DNA was added dropwise into the EP tube containing the diluted transfection reagent, mixed well, and allowed to stand at room temperature for 15 min. The mixture of the plasmid and the transfection reagent was added into the culture solution containing 293T cells in a 10 cm dish, mixed well, and cultured in an incubator at 37° C., 5% CO₂, and 95% relative humidity. The viral supernatant was collected 48 h and 72 h after cell transfection (with fresh medium replaced 48 h after transfection and after the viral supernatant was collected), filtered with a 0.45 μm filter to obtain lentivirus overexpressing IMTP (LV-IMTP+LAMP2B) and empty vector lentivirus (LV-Blank+LAMP2B), respectively, The viral preparations were then stored in a −80° C. refrigerator after aliquoted.

ii) Lentivirus infection: the naive hUC-MSCs in step (1) were passaged to the logarithmic growth phase and infected with LV-IMTP+LAMP2B and LV-Blank+LAMP2B separately, with a virus titer of 1*10⁸ tu/mL and an MOI of 50, where 50 μL of virus solution was added into per 6 mL of cell solution (with a cell concentration of 10⁵ cells/mL), and a mixture was mixed well and cultured at 37° C. with 5% CO₂. After 72 h of infection, hUC-MSCs overexpressing IMTP (denoted as IMTP-hUC-MSCs) and hUC-MSCs infected with empty vector lentivirus (denoted as Blank-hUC-MSCs) were obtained.

### Example 2

This example provides a method for preparing hUC-MSC-ES or the hUC-MSC-ES overexpressing the IMTP, including the following steps.

The subcultured P4 generation hUC-MSCs (cell concentration of 10⁵ cells/mL) were suspended in DMEM/F12 medium containing 10% FBS, cultured at 37° C., 5% CO₂, and saturated humidity for 72 h, and then transferred to fetal bovine serum (FBS)-containing exsome-free medium for another 48 h of culture. The collected cell culture supernatant was filtered through a 0.22 μm filter membrane. Large vesicles were removed by centrifugation at 2,000 g for 30 min at 4° C. and then at 10,000 g for 45 min at 4° C. The resulting supernatant was filtered through a 0.45 μm filter membrane (Millipore, USA). The filtrate was collected, and ultracentrifuged at 4° C. and 100,000 g for 70 min in an ultraspeed rotor. The pellet was resuspended in pre-cooled PBS, and then ultracentrifuged again at 4° C. and 100,000 g for 70 min. After the pellet was resuspended in 10 mL of PBS, the supernatant was collected to obtain the ES solution, which was stored at −4° C. for later use on a same day.

Three groups of hUC-MSCs, namely, naive hUC-MSCs, IMTP-hUC-MSCs and Blank-hUC-MSCs prepared in Example 1, were used to prepare three groups of ES solutions according to the above preparation method, and the concentration of the ES solutions was 2 mg/mL. The ES prepared from naive hUC-MSCs, Blank-hUC-MSCs, and IMTP-hUC-MSCs were named control-ES, Blank-ES, and IMTP-ES, respectively. The three types of ES were aliquoted and stored at −80° C. for subsequent analysis.

### Example 3

This example provides a preservation solution, a colloidal solution, and a preservation method for the hUC-MSC-ES for long-term storage of ES. The preservation solution included 20% (w/v) plant-derived recombinant human serum albumin (purchased from Healthgen Biotechnology, specification 10 g), 30 g/L trehalose, 50% (v/v) glycerol, 3% (mass fraction) ectoine, and 0.01 mol/L PBS, and the preservation solution had a pH value of 7.2. A method for preparing the preservation solution included the followings. The above components were mixed at room temperature, dissolved by stirring, and filtered with a 0.22 μm filter membrane for later use.

A method for preparing the colloidal solution included the followings. The preservation solution was heated at 90° C. for 20 min to form the colloidal solution, and then cooled.

The preservation method included the following steps. The colloidal solution and a solution of the hUC-MSC-ES were mixed at a volume ratio of 1:1 and then aliquoted and stored. The hUC-MSC-ES could be stably stored at −20° C. for 1 month; and the hUC-MSC-ES could be stably stored at −80° C. without freezing and thawing for 6 months.

### Experimental Example 1

### 1. Identification of hUC-MSCs

The hUC-MSCs cultured in step (1) of Example 1 were identified by the following methods: (1) cell morphology was observed under an inverted phase contrast microscope; (2) growth curves were recorded; (3) CD105, CD73, CD90, CD34, CD45, and HLA-DR were detected by flow cytometry; and (4) osteogenic differentiation and adipogenic differentiation analysis were conducted by Alizarin Red staining and Oil Red O staining, respectively.

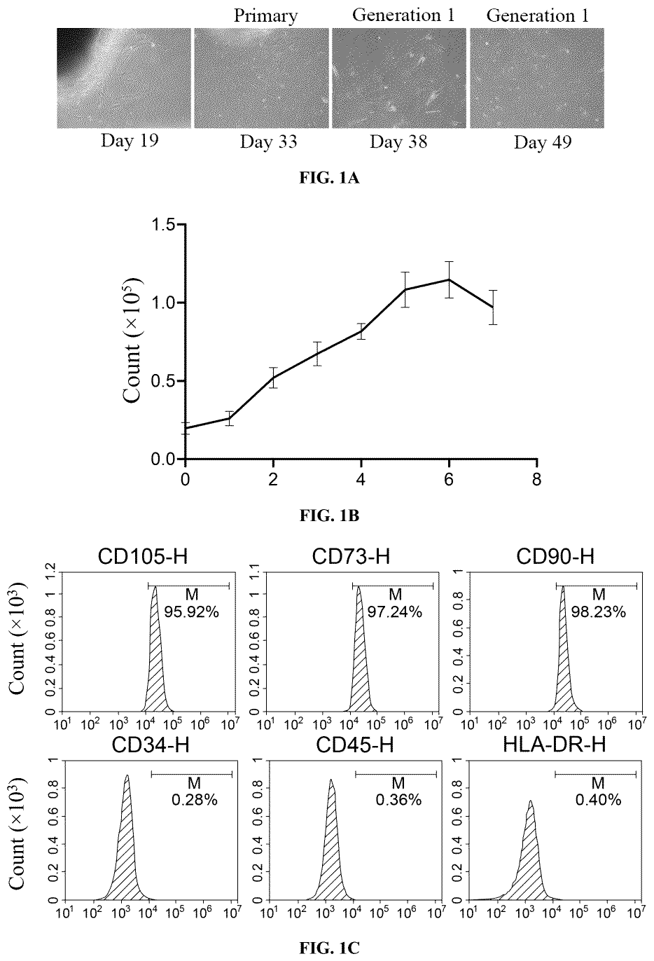

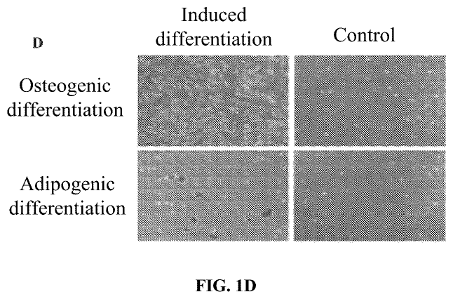

FIG. 1 shows the identification results of hUC-MSCs, and FIG. 1A and FIG. 1B show that the cells grew well. The flow cytometry results in FIG. 1C showed that CD105, CD73, and CD90 expressions were positive, while CD34, CD45, and HLA-DR expressions were almost negative, which were consistent with the characteristics of hUC-MSCs. FIG. 1D show that after the addition of adipogenic induction solution, oil red O staining showed lipid fluid in the cells at 14 d, confirming the ability of hUC-MSCs to differentiate into adipocytes. After adding osteogenic induction solution, hUC-MSCs transformed into polygons around 7 d, and calcium nodules appeared around 14 d. Alizarin red staining confirmed that hUC-MSCs had the ability of hUC-MSCs to differentiate into osteoblasts (FIG. 1D).

### 2. Identification of Myocardium-Targeted Engineered ES (IMTP-ES)

The three groups of hUC-MSCs in Example 1 or the three groups of ES in Example 2 were detected as follows: (1) the infection efficiency of hUC-MSCs was detected by fluorescence microscopy; (2) the expression levels of Lamp2b and IMTP in the three groups of hUC-MSCs were detected by RT-PCR; (3) the expression levels of Lamp2b and IMTP in the three groups of hUC-MSCs were detected by Western blot; (4) the expression levels of Lamp2b and IMTP in the three groups of ES were detected by RT-PCR; (5) the expression levels of Lamp2b and IMTP in the three groups of ES were detected by Western blot.

The primer sequences, PCR system, and PCR program in the PCR identification method are shown in Tables 1 to 3. The PCR amplification products were detected by gel electrophoresis, and the agarose gel concentration was 2%.

Western Blot identification included: protein extraction, quantification, and Sodium Dodecyl Sulfate-Polyacrylamide Gel Electrophoresis (SDS-PAGE) to separate proteins after sample loading. The proteins were transferred to Polyvinylidene Fluoride (PVDF) membrane under a constant current. The membrane was blocked with 5% skim milk powder, incubated with primary antibody at 4° C. overnight followed by recovering of the primary antibody, and incubation with secondary antibody at 37° C. for 45 min. The membrane was luminesced with substrate chemiluminescence kit, followed by color development and fixation. The conditions for primary and secondary antibody incubation are shown as follows, and antibodies used were purchased from ABclonal and wanleibio.

TABLE 1
Primary antibody incubation conditions

| Primary antibody | Dilution rate | Incubation conditions |
|---|---|---|
| LAMP2B antibody | 1:1000 | 4° C. overnight |
| β-actin antibody | 1:1000 | 4° C. overnight |

TABLE 2
Secondary antibody incubation conditions

| | Secondary | Dilution | Incubation |
|---|---|---|---|
| Primary antibody | antibody name | rate | conditions |
| LAMP2B antibody | Goat anti-rabbit | 1:5000 | 37° C. 45 min |
| | IgG-HRP | | |
| β-actin antibody | Goat anti-rabbit | 1:5000 | 37° C. 45 min |
| | IgG-HRP | | |

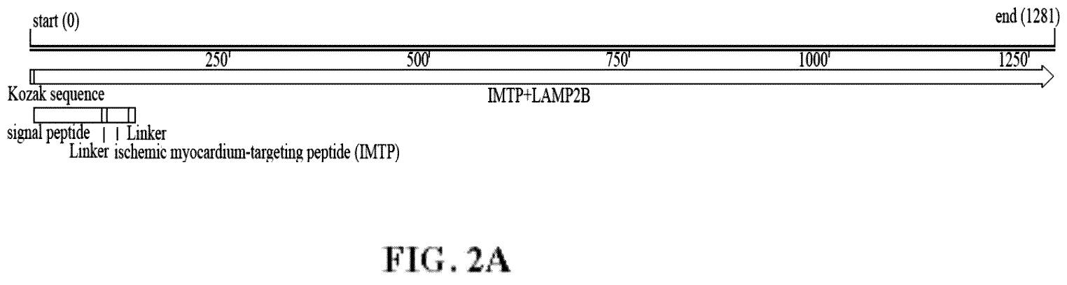

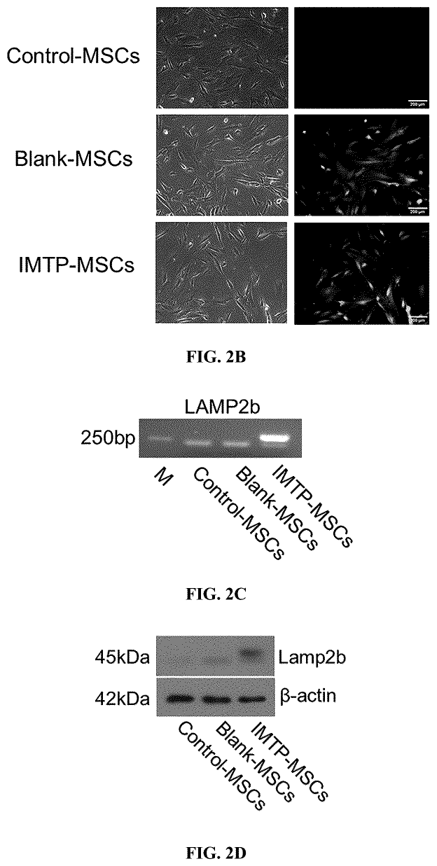

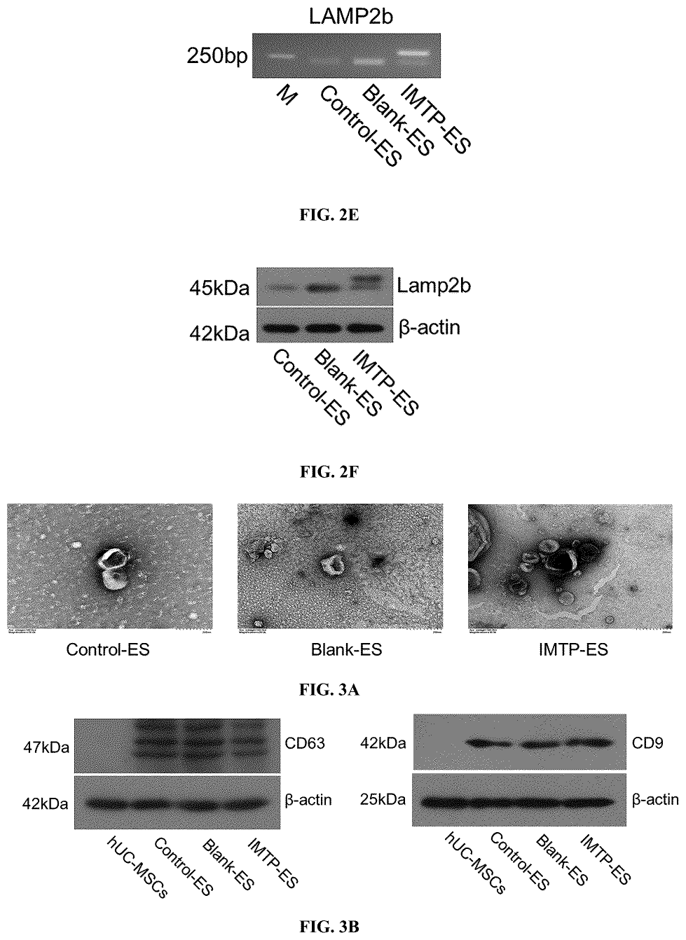

The results are shown in FIG. 2, where FIG. 2A was a schematic diagram of LAMP2b protein fused to IMTP sequence. FIG. 2B showed that clear green fluorescence signals were present in both IMTP-hUC-MSCs (IMTP-MSCs group) and Blank-hUC-MSCs (Blank-MSCs group), and infection efficiencies of the lentivirus overexpressing IMTP and the empty vector lentivirus to the hUC-MSCs were 85.2% and 85.8%, respectively. This confirmed the successful transfection of hUC-MSCs by the lentivirus in IMTP-hUC-MSCs and Blank-hUC-MSCs. In FIG. 2C to FIG. 2F, PCR and Western blot results demonstrated that the mRNA and protein expression of Lamp2b+IMTP in the IMTP-ES group were significantly higher than those of Lamp2b+IMTP in Control-ES and Blank-ES, confirming the successful establishment of engineered ES (IMTP-ES) targeting the myocardium.

The three groups of hUC-MSCs in Example 1 or the three groups of ES in Example 2 were identified as follows: (1) the morphologies of the three groups of ES were observed under TEM. (2) Particle size distribution of three groups of ES was measured based on nanoparticle tracking analysis. (3) Using naive hUC-MSCs as a control, the expression levels of CD9 and CD63 in the three groups of ES were detected by Western blot.

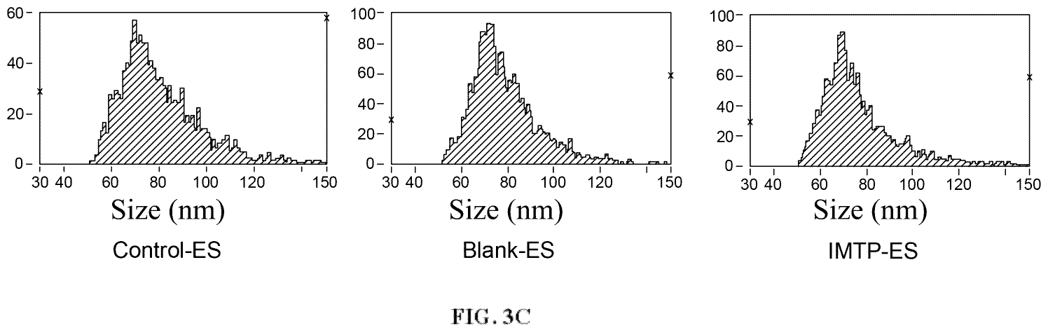

In FIG. 3, TEM observation showed that these extracted hUC-MSC-ES cells had typical cup-shaped spherical vesicles (FIG. 3A). The results of particle size analysis showed that the average particle size of naive ES (Control-ES) from hUC-MSCs not infected with lentivirus was 79.26±14.79 nm, while the average particle sizes of ES from Blank-MSCs and IMTP-MSCs were 78.44±13.23 nm and 75.88±14.21 nm, respectively, proving that there was no significant difference in the particle size distribution of the three ES (FIG. 3C). Western Blot analysis showed that the expression of specific surface markers CD63 and CD9 in the three types of ES was significantly higher than that in the parental cells (FIG. 3B). There were no significant differences between the myocardium-targeting engineered ES and native hUC-MSC-ES in morphology, size, and surface markers.

### Experimental Example 2

### 1. Cell Treatment and Grouping of Medication

Establishment of Hypoxia/Reoxygenation Model for hESC-CMs and HEK293 Cells:

The hESC-CMs were quickly dissolved in a 37° C. water bath to revive the cells, which were centrifuged at 150 g for 3 min. The pellet was resuspended in RPMI-1640 medium containing 10% FBS+B27, mixed and inoculated into a 6-well culture plate, and cultured at 37° C. in a 5% CO₂ incubator. Human embryonic kidney cell line HEK293 was cultured in DMEM medium containing 10% FBS and 1% P/S at 37° C. in a 5% CO₂ incubator. After cultured for 24 h, the cells was observed and the medium was changed. The cells were washed with PBS and then transferred to fresh medium for culture. Trypsin was added for digestion and passaging.

The cells were adjusted to the logarithmic growth phase, counted, and inoculated into a 96-well culture plate, each well being 3×10³ cells, and 5 replicate wells were designed for each group. The cells were randomly divided into the following six groups. Control group: cells were cultured without any treatment. H/R group: only hypoxia/reglycation and reoxygenation treatment was conducted, without the addition of ES or PBS. When the cell confluence reached 70%, the medium was replaced with the corresponding glucose-free medium, and the cells were cultured in a hypoxic (5% CO₂, 95% N₂) incubator. After 4 h of hypoglycemia and hypoxia, the complete medium was replaced with reglycation and reoxygenation culture for 6 h. H/R+PBS group: when the cell confluence reached 70%, the medium was replaced with the corresponding glucose-free medium, and the cells were cultured in a hypoxic (5% CO₂, 95% N₂) incubator. After 4 h of hypoglycemia and hypoxia, the complete medium was replaced. To each well, 50 μL PBS was added, and the cells were subjected to reglycation and reoxygenation and cultured for 6 h. H/R+ES group: when the cell confluence reached 70%, the medium was replaced with the corresponding glucose-free medium, and the cells were cultured in a hypoxic (5% CO₂, 95% N₂) incubator. After 4 h of hypoglycemia and hypoxia, the complete medium was replaced, Afterwards, 100 μg of the three different types of ES obtained in Example 2, i.e., Control-ES, Blank-ES, and IMTP-ES, were added into each well, respectively, subjected to reglycation and reoxygenation, and cultured for 6 h.

### 2. Test Method

### (1) Detection of Cell Viability by CCK-8 Method

TABLE 3
Main instruments

| Instrument | Instrument model | Locality | Company |
|---|---|---|---|
| Microplate reader | 800Ts | U.S. | BIOTEK |

TABLE 4
Main reagents and manufacturers thereof

| Reagent | Cat. No. | Manufacturer | Locality |
|---|---|---|---|
| CCK-8 | WLA074 | Wanleibio | China |

CCK-8 assay: the supernatant of cells in each group was discarded, 100 μL of complete medium was added to each well; 10 μL of CCK-8 solution was added to each well and cultured in an incubator at 37° C., 5% CO₂ for 1 h; the OD value of each well at 450 nm was detected on a microplate reader for data analysis.

### (2) Detection of Total Superoxide Dismutase (T-SOD) Activity and Malondialhyde (MDA) Level by Chemical Method

TABLE 5
Main reagents for SOD detection and manufacturers thereof

| Reagent | Manufacturer | Cat. No. | Locality |
|---|---|---|---|
| BCA protein concentration | Wanleibio | WLA004 | China |
| assay kit | | | |
| Superoxide dismutase (SOD) | Wanleibio | WLA110 | China |
| assay kit | | | |

SOD detection: the SOD activity in the supernatant of each group of cells was determined by a method in the instructions of the detection kit. A test tube and a control tube were set up. In the test tube, 0.2 mL of sample was added, and an equal amount of double distilled water was added to the control tube. The samples were fully mixed, placed in a constant-temperature gas bath at 37° C. for 40 min. Then, 2 mL of the color developer was added, mixed, and placed at room temperature for 10 min. The colorimetry was conducted at a wavelength of 550 nm, a light path of 1 cm, and distilled water was used for zeroing. SOD activity (U/mgprot)=((control OD value−measured OD value)/control OD value)/50%×(total volume of reaction solution (mL)/sampling volume (mL))/protein concentration of sample to be tested (mgprot/mL).

TABLE 6
Main reagents for MDA detection and manufacturers thereof

| Reagent | Manufacturer | Cat. No. | Locality |
|---|---|---|---|
| Malondialdehyde (MDA) assay kit | Wanleibio | WLA048 | China |
| BCA protein concentration assay | Wanleibio | WLA004 | China |
| kit | | | |

MDA detection: the MDA content in the supernatant of each group of cells was determined following the instructions of the detection kit. Standard tube, blank tube, measurement tube, and control tube were set up. The samples and reagents were added, vortexed to mix well. The mouth of the test tube was tied with plastic wrap, and a small hole was pierced with a needle. The tubes were boiled in 95° C. water bath for 40 min. The test tube was cooled, and then centrifuged at 4,000 r/min for 10 min. The absorbance of the supernatant was measured at a wavelength of 532 nm, a light path of 1 cm, and distilled water was used for zeroing. MDA content (nmol/mgprot)=((measured OD value−control OD value)/(standard OD value−blank OD value))×standard tube concentration (10 nmol/mL)/tested homogenate protein concentration (mgprot/mL).

### (3) Detection of TNF-α, IL-1β, and IL-10 Levels in the Cell Supernatant of Each Group Using Enzyme-Linked Immunosorbent Assay (ELISA)

TABLE 7
Main reagents and manufacturers thereof

| Reagent | Company | Cat. No. | Locality |
|---|---|---|---|
| Tumor necrosis factor-α kit | Wanleibio | WLE05 | China |
| (TNF-α) ELISA assay | | | |
| Interleukin-6 (IL-6) | Wanleibio | WLE04 | China |
| ELISA assay kit | | | |
| Interleukin-1β (IL-1β) | Wanleibio | WLE03 | China |
| ELISA assay kit | | | |

ELISA: the levels of TNF-α, IL-1β, and IL-10 in the supernatant of each group were determined following the instructions of the assay kit, and the TNF-α, IL-6, IL-1β standards, capture antibodies, horseradish peroxidase (HRP)-Streptavidin, 3,3′,5,5′-Tetramethylbenzidine (TMB) colorimetric solution, and 10× coating solution were diluted and incubated with the corresponding staining solution antibodies at 4° C. overnight. 100 μL of each diluted standard was added into a 96-well plate, 1×PBST was added to the blank well, and incubated at 37° C. for 2 h. 100 μL of diluted capture antibody was added and reacted at 37° C. for 1 h. The product was washed 3 times with 300 μL Phosphate Buffered Saline with Tween-20 (PBST) as washing solution, immersed for 2 min each time. 100 μL of diluted HRP-Streptavidin was added for reaction at 37° C. for 30 min. The product was washed 5 times with 300 μL PBST Washing Buff, immersed for 2 min each time. 100 μL of TMB colorimetric solution was added to each well for reaction at 37° C. for 15 min, and 50 μL of TMB stop solution D was added to terminate the reaction. The absorbance at 450 nm was read using a microplate reader. With the standard concentration (pg/mL) as the ordinate and the corresponding optical density (OD) as the abscissa, a linear regression curve of the standard was plotted, and the concentration value of each sample was calculated according to the curve equation.

### (4) Detection of Bax, Cleaved-Caspase3, and FasL Expression by Western Blot

TABLE 8
Primary antibody incubation conditions

| Primary antibody | Dilution rate | Incubation conditions |
|---|---|---|
| Bax antibody | 1:500 | 4° C. overnight |
| caspase3/cleaved- | 1:1000 | 4° C. overnight |
| caspase³ antibody | | |
| FasL antibody | 1:500 | 4° C. overnight |
| β-actin antibody | 1:1000 | 4° C. overnight |

TABLE 9
Secondary antibody incubation conditions

| Primary | Secondary | Dilution | Incubation |
|---|---|---|---|
| antibody | antibody | rate | conditions |
| Bax antibody | Goat anti-rabbit IgG-HRP | 1:5000 | 37° C. 45 min |
| caspase3/cleaved- | Goat anti-rabbit IgG-HRP | 1:5000 | 37° C. 45 min |
| caspase3 antibody | | | |
| FasL antibody | Goat anti-rabbit IgG-HRP | 1:5000 | 37° C. 45 min |
| β-actin antibody | Goat anti-rabbit IgG-HRP | 1:5000 | 37° C. 45 min |

### (5) Detection of Apoptosis Level of Each Group of Cells by Flow Cytometry

TABLE 10
Kit

| Reagent | Cat. No. | Manufacturer | Locality |
|---|---|---|---|
| Apoptosis assay kit | KGA108 | KeyGEN | China |
| | | BioTECH | |

According to the method given in the instructions of the assay kit, each group of cells was centrifuged at 1,000 rpm for 5 min and washed twice with PBS. The cells were resuspended in 500 μL of Binding Buffer, 5 μL of Annexin V-FITC was added and mixed well, and 5 μL of Propidium Iodide was added and mixed well. The cells were incubated in the dark at room temperature for 15 min, and then subjected to flow cytometry.

(6) Fluo-3/AM Fluorescent Probe Staining and Confocal Microscopy Observation of Free Ca²⁺ ([Ca²⁺]i) in Each Group of Cells

Each group of cells was centrifuged at 1,000 rpm for 5 min, and the supernatant was discarded. The cells were washed twice with PBS, mixed with diluted Fluo-3AM staining solution, and cultured in a 37° C. incubator for 1 h. After centrifugation, the cells were washed 3 times with PBS and observed under a laser scanning confocal microscope.

TABLE 11
Main reagents and manufacturers thereof

| Reagent | Cat. No. | Manufacturer | Locality |
|---|---|---|---|
| Fluo-3AM | ab145254 | abcam | China |

(7) Staining with Fluo-3/AM Fluorescent Probe and Flow Cytometry Observation of Free ([Ca²⁺]i) in Each Group of Cells

Each group of cells was centrifuged at 1,000 rpm for 5 min, and the supernatant was carefully removed. The cells were washed twice with PBS, mixed with diluted Fluo-3AM staining solution, and cultured in a 37° C. incubator for 1 h. After centrifugation, the cells were washed 3 times with PBS and subjected to flow cytometry.

### 3. Test Results

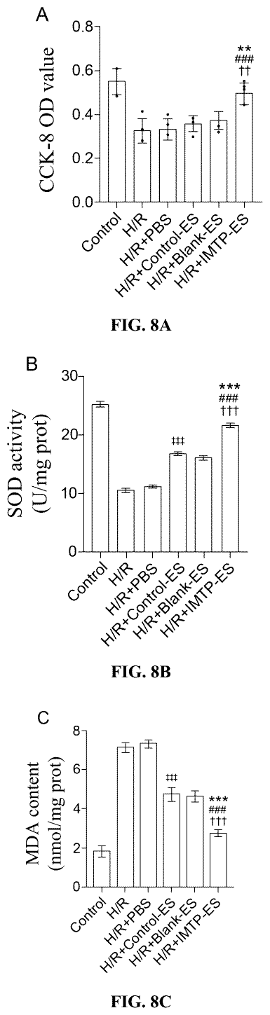

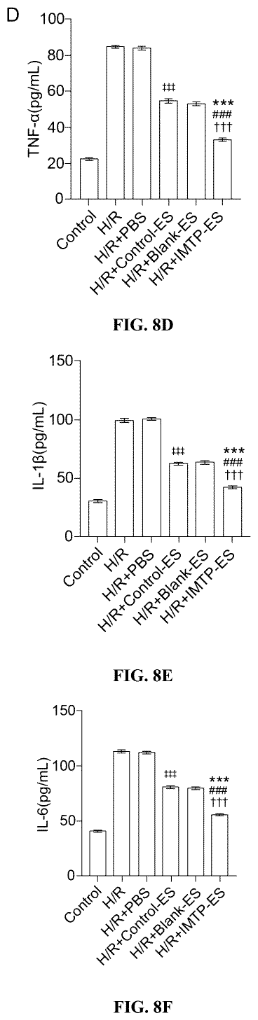

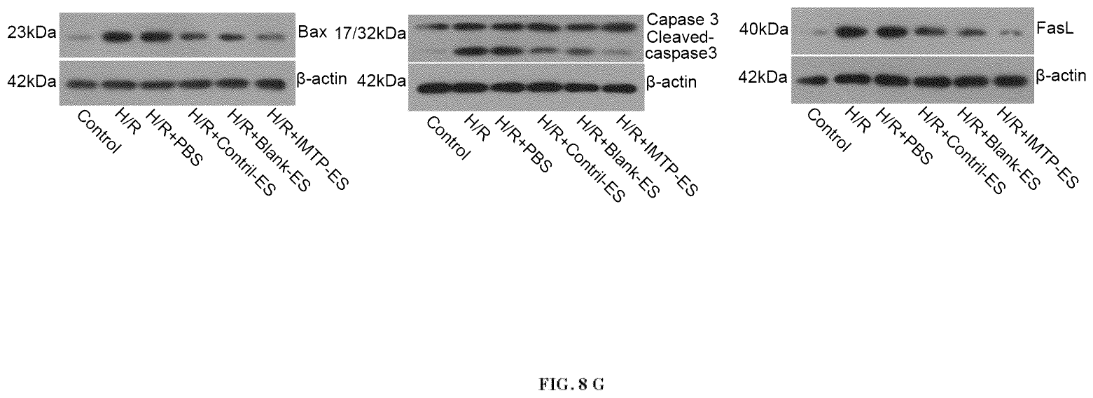

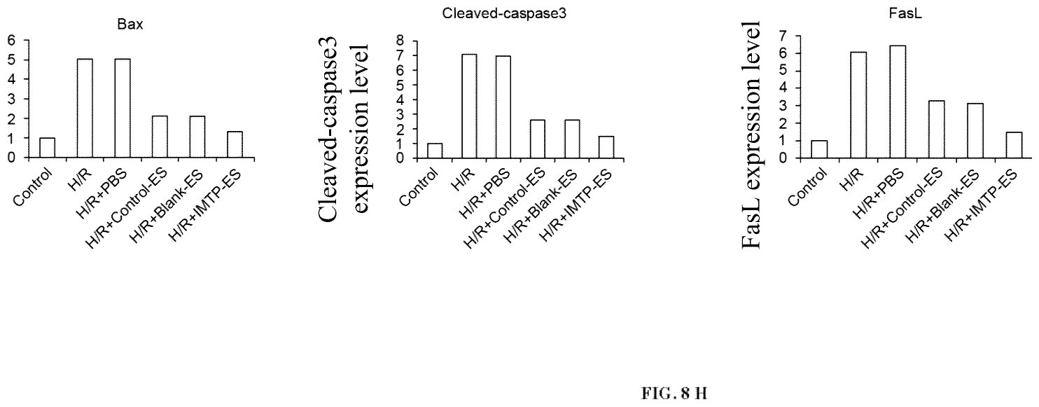

As shown in FIG. 8, compared with those of the H/R group and H/R+PBS group, the survival of CMs in the three hUC-MSC-ES treated groups was significantly increased, the T-SOD activity in the cells was enhanced, the MDA level was decreased, the apoptosis was reduced, and the expression of apoptotic proteins Bax, cleaved-caspase3, and FasL was reduced (p<0.05). Compared with that of the H/R+Control-ES group and the H/R+Blank-ES group, the survival of CMs in the H/R+IMTP-ES group was significantly increased, the T-SOD activity in the cells was enhanced, the MDA level was significantly decreased, the apoptosis was significantly reduced, and the expression of apoptotic proteins Bax, cleaved-caspase3, and FasL was significantly reduced (p<0.05). ELISA showed that the levels of TNF-α, IL-1β, and IL-6 in the supernatant of CMs in the hUC-MSC-ES treatment group were significantly reduced (p<0.05). The levels of TNF-α, IL-1β, and IL-6 in the supernatant of CMs in the H/R+IMTP-ES group were further reduced compared with those in the H/R+Control-ES group and the H/R+Blank-ES group (p<0.05).

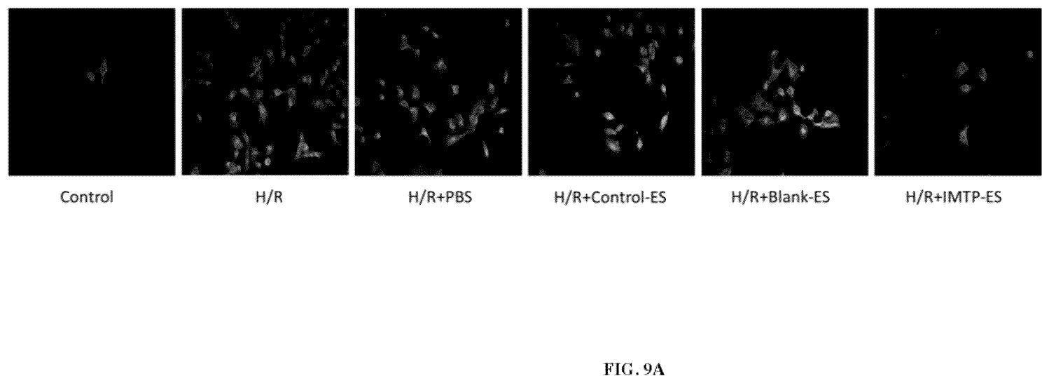

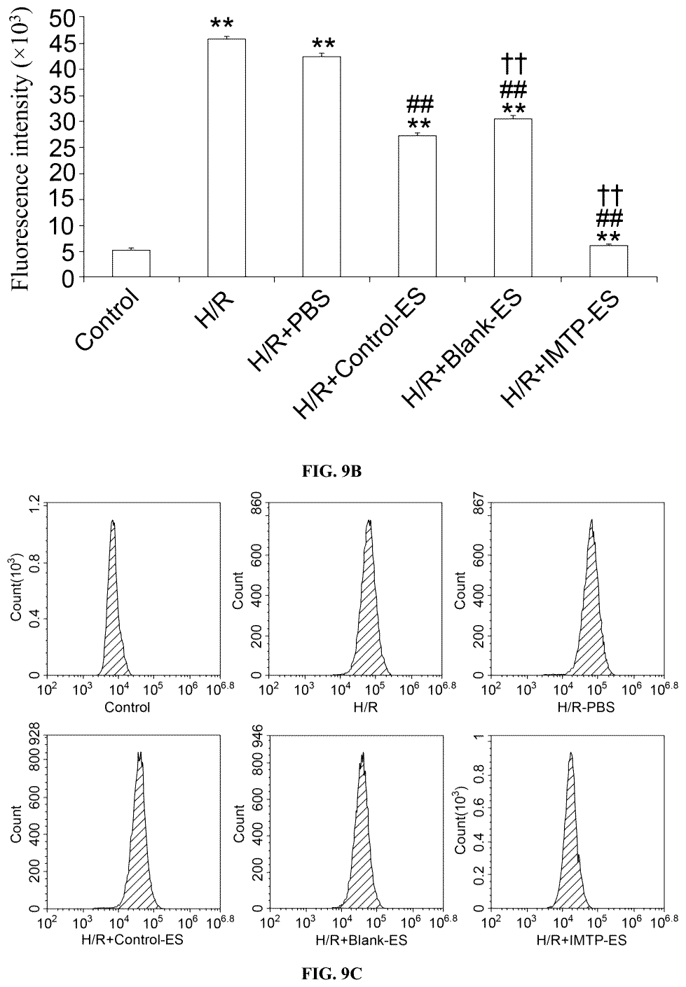

As shown in FIG. 9, Fluo-3/AM fluorescent probe staining and confocal microscopy showed that the free Ca²⁺ in CMs of the hUC-MSC-ES treatment group was significantly reduced compared with that of the H/R group and H/R+PBS group. Flow cytometric analysis showed that the free Ca²⁺ concentration was reduced in CMs of the three hUC-MSC-ES treatment groups. Compared with that of the H/R+PBS group, the free Ca²⁺ concentration in the H/R+Control-ES group, the H/R+Blank-ES group, and the H/R+IMTP-ES group reduced 40.4%, 41.0%, and 73.7%, respectively.

The intracellular free Ca²⁺ concentration in the CMs of the H/R+IMTP-ES group was significantly reduced compared with that of the H/R+Control-ES group and the H/R+Blank-ES group.

### Experimental Example 3 Tubule Formation Test

### 1. Cell Treatment and Grouping of Medication

Human Umbilical Vein Endothelial Cells (HUVECs) were cultured in an endothelial cell medium containing 5% fetal bovine serum and 1% endothelial cell culture supplement at 37° C. in a 5% CO₂ incubator.

TABLE 12
Main reagents and manufacturers thereof

| Reagent | Cat. No. | Manufacturer | Locality |
|---|---|---|---|
| Endothelial cell culture | PriMed-iCell-002 | iCell Bioscience | China |
| system | | Inc | |
| Matrigel | 356234 | corning | U.S. |

Cell culture in tubes: the Matrigel was thawed at 4° C. overnight, and pipetted onto a 96-well culture plate, with 50 μL per well. The plate was incubated in a 37° C. incubator for 1 h to solidify. HUVECs were collected and inoculated at a density of 5×10⁴ cells in a 24-well plate pre-coated with Matrigel, and PBS or the three ES obtained in Example 2 were added according to the following grouping. The 24-well plate was placed in a cell culture incubator at 37° C., 5% CO₂, and saturated humidity for 4 h, 6 h, and 12 h, and observed and photographed under an inverted phase contrast microscope. Image analysis software was used to count the number of tubule branch nodes and measure the cumulative tubule length and the number of tubule loops formed in each group.

### Experimental Grouping

A. HUVEC+PBS group;
B. HUVEC+50 μg/mL Control-ES group;
C. HUVEC+50 μg/mL Blank-ES group;
D. HUVEC+50 μg/mL IMTP-ES group.

### 2. Experimental Results

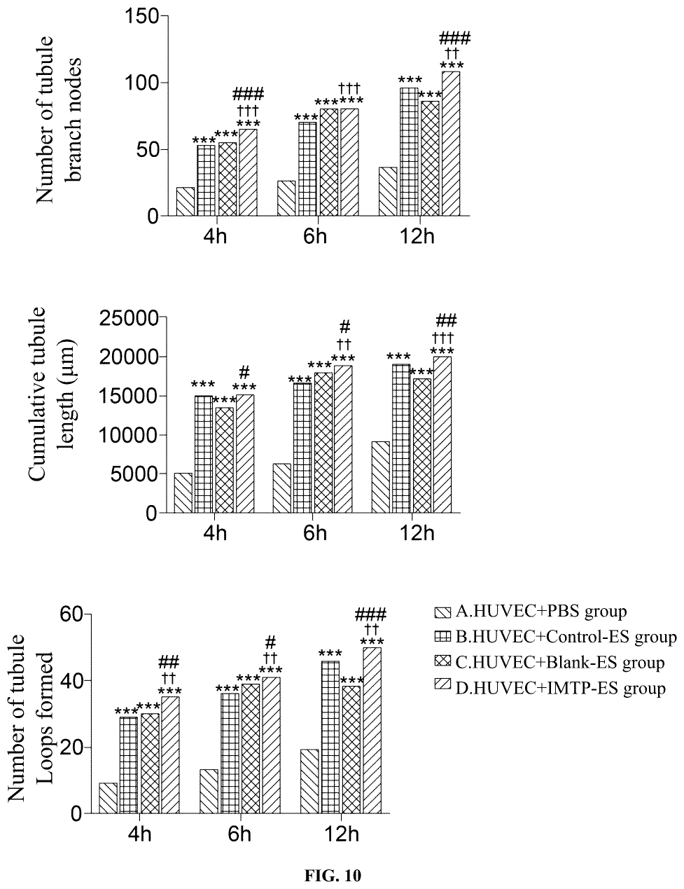

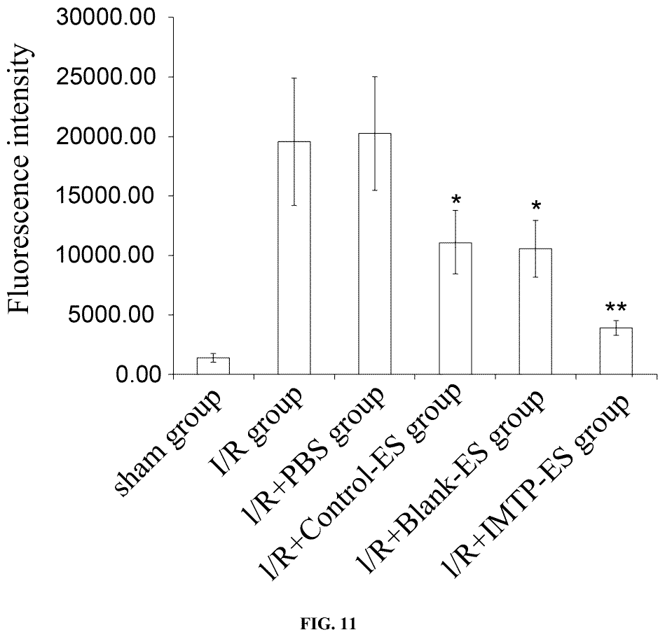

In FIG. 10, the results of the tubule formation experiment showed that hUC-MSC-ES treatment significantly increased the number and length of tubules generated by HUVECs and the number of tubule loops formed, suggesting that hUC-MSC-ES could promote angiogenesis of HUVECs in vitro.

### Experimental Example 4

### 1. Experimental Method

A total of 18 rats (Sprague-Dawley (SD) rats, male, 8-10 weeks, 250-300 g) were randomly divided into I/R model, Sham group (sham operation group), I/R+PBS group, I/R+Control-ES group, I/R+Blank-ES group, and I/R+IMTP-ES group, 3 rats in each group. Except for the sham operation group, the MIRI model of rats was established by ligation of the anterior descending branch. Specifically, male SD rats were acclimatized for 1 week and placed supine on a heating pad at 37±3° C. after anesthesia. After the small animal ventilator was turned on, the respiratory rate was set to 60-70 times/min, and the rat was connected to the small animal ventilator for assisted breathing after tracheal intubation. The left thorax of the rat was opened to expose the heart at the third intercostal space, the pericardium was separated, and the heart was extruded. The left anterior descending coronary artery was ligated with 6-0 suture, with a width and depth of both approximately 2 mm. The color below the area to be ligated turned white and the electrocardiogram showed S-T segment elevation in the precordial leads, indicating that the ligation of the left anterior descending coronary artery was successful. After 30 min of ischemia, the sutures were removed to initiate the reperfusion, thus establishing the I/R model.

After the I/R model was established, the I/R+Control-ES, I/R+Blank-ES, and I/R+IMTP-ES groups were injected once with three types of ES obtained in Example 2 (Control-ES, Blank-ES, IMTP-ES) via the tail vein at 400 μg/200 μL. I/R+PBS group: after the I/R model was established, an equal volume of PBS solution (concentration 0.01 M, pH=7.3) was injected into the tail vein. The I/R group received no treatment. The sham operation group only had thread hanging but no ligation.

After 24 h of the above treatment, the rats were anesthetized by intraperitoneal injection of 2% sodium pentobarbital (0.25 mL/100 g) according to their body weight. Their heart was exposed by opening the thorax, and 0.2 mL of 1% sodium heparin was injected into the left ventricle. The aorta was cannulated through the left ventricle, and the right atrial appendage was cut open. The blood vessels were quickly flushed with 150 mL of physiological saline, and then perfused and fixated with 250 mL of 0.01 mol/L PBS (4° C., pH=7.40) containing 4% paraformaldehyde. After that, the heart tissue at the infarction border zone of MI was fixated in 4% paraformaldehyde and stored in a refrigerator at 4° C.

### 2. Test Method

### (1) Expression of MCP-1, IL-1β, and TNF-α Detected by Western Blot

The cardiac tissues from the infarction border zone of rats in each group mechanically homogenized in physiological saline at a weight (g) to volume (mL) ratio of 1:9, under ice-water bath, followed by centrifugation at 2,500 rpm for 10 min, and the supernatant was taken for measurement. The following antibodies were used to allow primary antibody incubation, recovery of the primary antibody, secondary antibody incubation, and development to detect the expression of MCP-1, IL-1β, and TNF-α.

TABLE 13
Primary antibody incubation conditions

| Primary antibody | Dilution rate | Incubation conditions |
|---|---|---|
| TNF-α antibody | 1:500 | 4° C. overnight |
| IL-1β antibody | 1:500 | 4° C. overnight |
| MCP I antibody | 1:500 | 4° C. overnight |
| β-actin antibody | 1:1000 | 4° C. overnight |

TABLE 14
Secondary antibody incubation conditions

| Primary | Secondary | Dilution | Incubation |
|---|---|---|---|
| antibody | antibody name | rate | conditions |
| TNF-α antibody | Goat anti-rabbit IgG-HRP | 1:5000 | 37° C. 45 min |
| IL-1β antibody | Goat anti-rabbit IgG-HRP | 1:5000 | 37° C. 45 min |
| β-actin antibody | Goat anti-rabbit IgG-HRP | 1:5000 | 37° C. 45 min |
| MCP I antibody | Goat anti-rabbit IgG-HRP | 1:5000 | 37° C. 45 min |

### (2) Oxidative Stress Level and Inflammatory Cell Infiltration

### Main Reagents and Manufacturers Thereof

| Reagent | Cat. No. | Manufacturer | Locality |
|---|---|---|---|
| DHE kit | S0063 | Beyotime | China |

Dihydroethidium (DHE) staining: the detection was conducted following the manufacturer's instruction of the DHE kit. The cardiac tissue of the infarction border zone of MI was placed on the sample holder, the sample was embedded with Optimal Cutting Temperature (OCT) embedding medium, and the sample was placed on a freezing microtome for precooling. The tissue was cut into 10 μm sections, and adsorbed onto a glass slide gently and quickly. After dried, the sections were stored in a −70° C. refrigerator. The sections were taken out, dried, and washed with distilled water 3 times, 5 min each time. DHE reagent was added, diluted at 1:100, and incubated at 37° C. in the dark for 30 min. The sections were taken out, immersed in PBS 3 times, 5 min each time. The sections were taken out one by one, a drop of anti-fluorescence quenching agent was added with a rubber-tipped dropper, and the sections were sealed with coverslips. The staining results were observed under a fluorescence microscope and photographed.

### (3) Observation of Tissue Morphological Changes Using H&E Staining

TABLE 15
Main reagents and manufacturers thereof

| Reagent | Cat. No. | Manufacturer | Locality |
|---|---|---|---|
| Hematoxylin | H8070 | Solarbio | China |
| Eosin Y | A600190 | sangon | China |

H&E staining: paraffin sections of cardiac tissue at the infarction border zone of MI of rats in each group were made, and sequentially placed in xylene I for 15 min-xylene II for 15 min-anhydrous ethanol I for 5 min-anhydrous ethanol II for 5 min-95% alcohol for 2 min-85% alcohol for 2 min-75% alcohol for 2 min-distilled water for washing. The sections were stained with hematoxylin for 5 min, immersed in distilled water for 5 min, differentiated in 1% hydrochloric acid alcohol for a few seconds, rinsed with running water for 20 min, and immersed in distilled water for 2 min. The sections were stained with eosin solution for 3 min. The sections were sequentially placed in 75% alcohol for 2 min-85% alcohol for 2 min-95% alcohol for 2 min-absolute ethanol I for 5 min-absolute ethanol II for 5 min-xylene I for 0 min-xylene II for 10 min for dehydration and transparency, and sealed with neutral gum. The sections were examined under a microscope and images are collected and analyzed.

### (4) Detection of Apoptosis in the Heart Tissue of Each Group of Rats by Terminal Deoxynucleotidyl Transferase Mediated Nick End Labeling (TUNEL) Staining

TABLE 16
Main reagents and manufacturers thereof

| Reagent | Cat. No. | Manufacturer | Locality |
|---|---|---|---|
| TUNEL kit (red fluorescence) | WLA127a | Wanleibio | China |

TUNEL staining: paraffin sections of cardiac tissue at the infarction border zone of MI of rats in each group were made, and sequentially placed in xylene I for 15 min-xylene II for 15 min-anhydrous ethanol for 5 min-anhydrous ethanol for 5 min-95% alcohol for 5 min-95% alcohol for 2 min-85% alcohol for 2 min-75% alcohol for 2 min-distilled water for 2 min-PBS for 5 min. 50 μL of 0.1% Triton X-100 was added dropwise, allowed to stand at room temperature for 8 min, and rinsed with PBS 3 times for 5 min each time. 50 μL of TUNEL reaction solution (prepared with enzyme solution and label solution at a ratio of 1:9) was added, incubated at 37° C. for 60 min, and rinsed with PBS 3 times for 5 min each time. DAPI was added dropwise, and the sections were counterstained for 5 min in the dark. The sections were washed with PBS and the staining results were observed under a microscope.

### (5) Detection of the Expression of TNF-α and CD206 in the Heart Tissue of Rats in Each Group Using Immunofluorescence Simple Staining, and Observation of the Polarization of Macrophages M1 and M2

Immunofluorescence simple staining: paraffin sections of cardiac tissue at the infarction border zone of MI of rats in each group were made, and sequentially placed in a 60° C. oven for 30 min-xylene I for 15 min-xylene II for 15 min-95% alcohol for 1 min-85% alcohol for 1 min-75% alcohol for 1 min, and rinsed with PBS 3 times, 5 min each time. The sections were placed in antigen retrieval solution and repaired at high temperature and low heat for 10 min, and then rinsed with PBS 3 times, each time for 5 min. 1% Bovine Serum Albumin (BSA) was added dropwise, and the sections were counterstained for 15 min at room temperature. The primary antibody was diluted with PBS at 1:100 and added dropwise until the tissue was completely covered. The tissue was incubated at 4° C. overnight and then rinsed with PBS 3 times, 5 min each time. The fluorescent secondary antibody was added, diluted with PBS at 1:200 until the tissue was completely covered. The tissue was incubated at room temperature for 60 min, and rinsed with PBS 3 times, 5 min each time. DAPI was added dropwise to counterstain the nucleus, and then rinsed with PBS 3 times, 5 min each time, and the slide was sealed with a coverslip. The staining results were observed under a fluorescence microscope.

TABLE 17
Main reagents and manufacturers thereof

| Reagent | Cat. No. | Manufacturer | Locality |
|---|---|---|---|
| TNF-α | AF7014 | Affinity | China |
| CD206 | DF4149 | Affinity | China |
| Cy3-labeled goat | A27039 | invitrogen | U.S. |
| anti-rabbit IgG | | | |
| BSA powder | A602440-0050 | Sangon Biotech | China |
| DAPI | D106471-5mg | Aladdin | China |
| Anti-fluorescence | S2100 | Solarbio | China |
| quenching agent | | | |

### (6) MDA Detection

MDA detection: the cardiac tissues from the infarction border zone of each group of MI were collected, standard tube, blank tube, measurement tube, and control tube were set up, samples and reagents were added, vortexed to mix well, and the tubes were boiled in 95° C. water bath for 40 min. The test tube was taken out and cooled with running water, and then centrifuged at 4,000 r/min for 10 min. The absorbance of the supernatant was measured at a wavelength of 532 nm.

### 3. Test Results

After 24 h of reperfusion, the CM apoptosis in the hUC-MSC-ES treatment group was significantly reduced (P<0.05, FIG. 11).

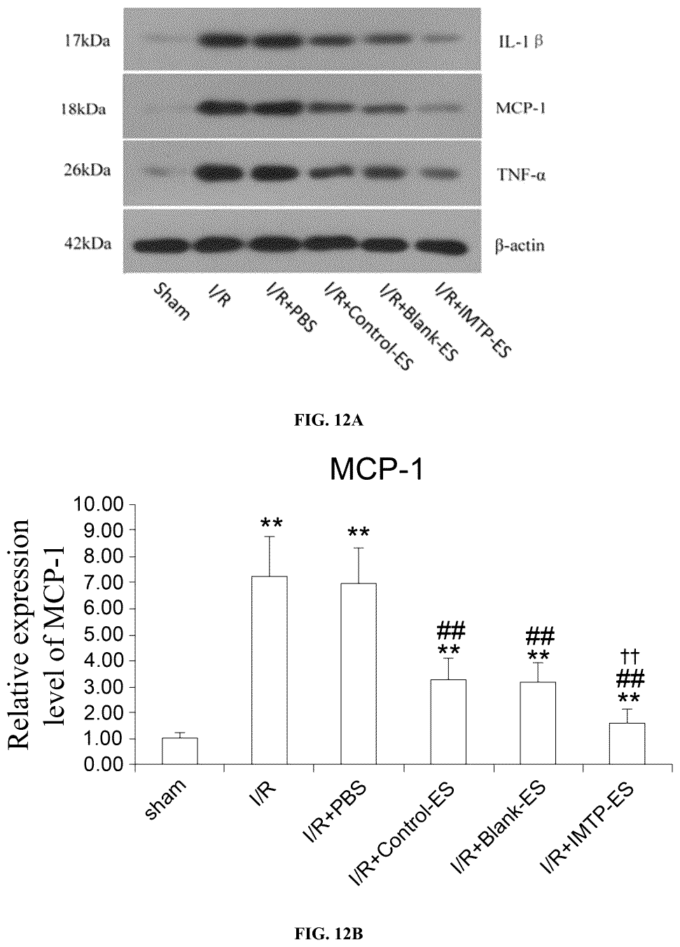

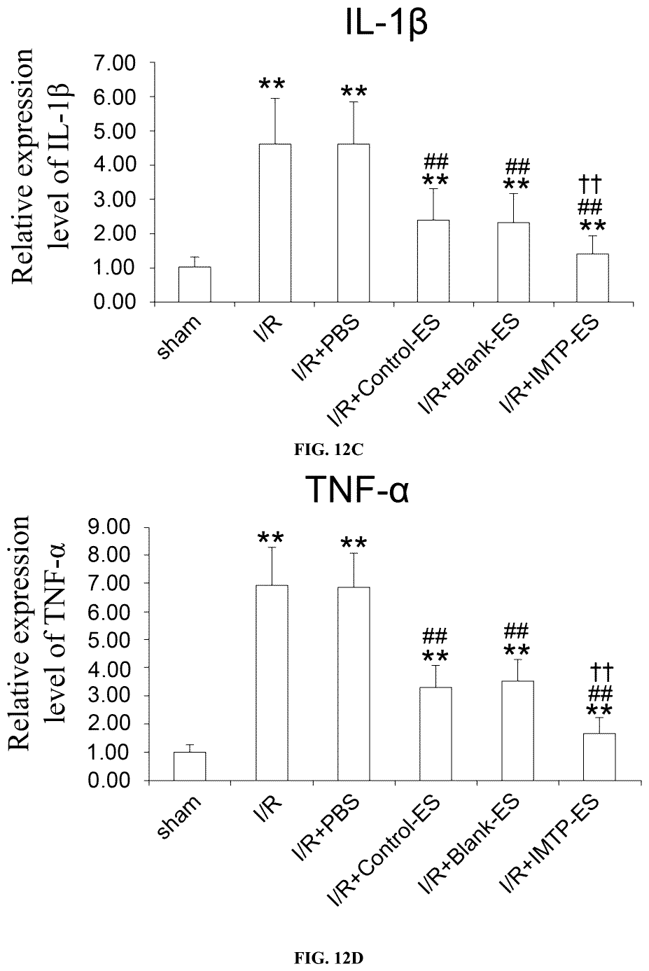

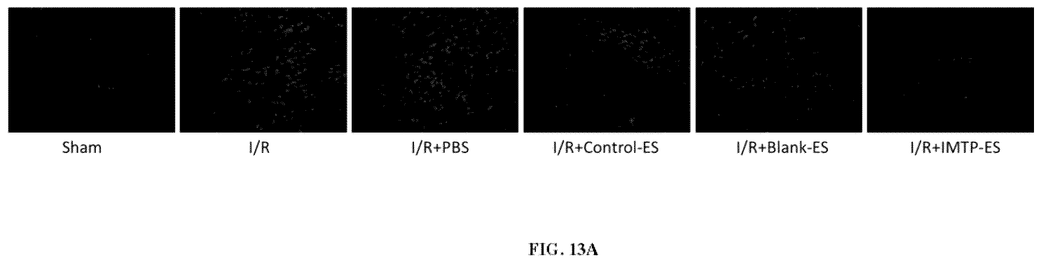

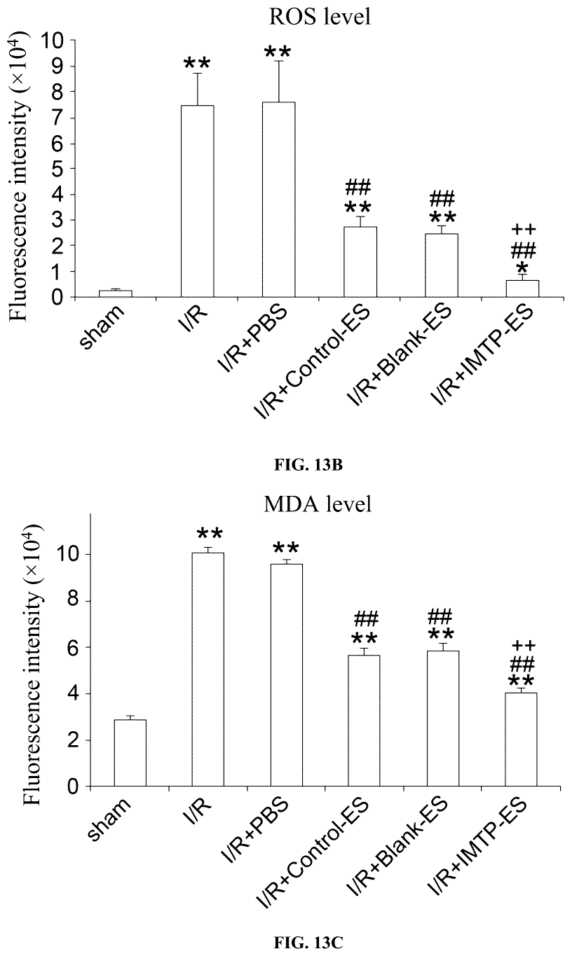

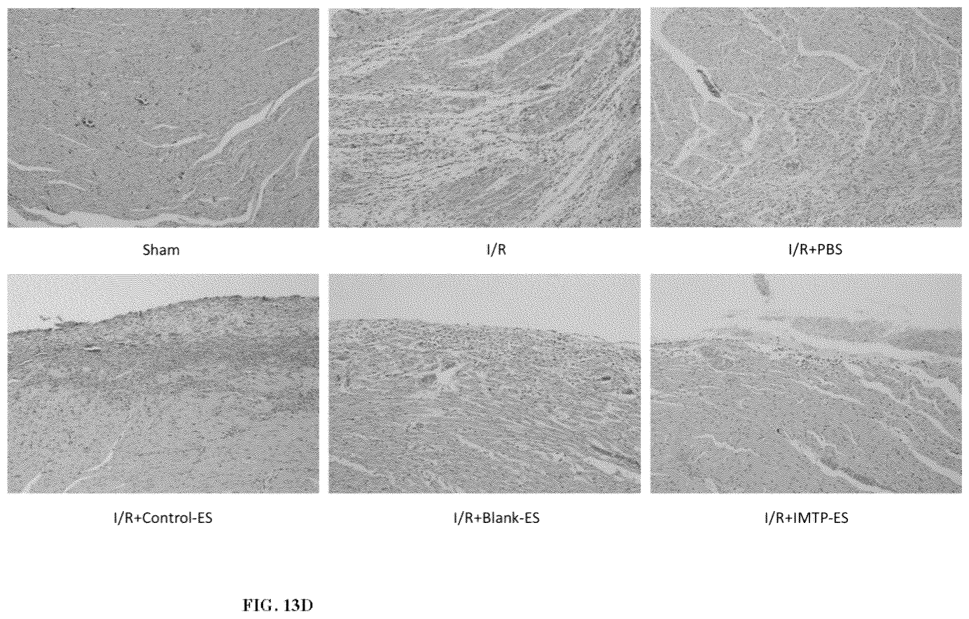

Western blot analysis showed that the expressions of TNF-α, IL-1β, and MCP-1 in the hUC-MSC-ES treatment group were significantly reduced (P<0.01, FIG. 12) compared with those of the I/R+PBS group. DHE fluorescent probe staining showed that ROS in the hUC-MSC-ES treatment group was significantly reduced (P<0.01), and the MDA level was significantly reduced (P<0.01). H&E staining analysis showed that the inflammatory cell infiltration in the hUC-MSC-ES group was alleviated (FIG. 13). These results suggested that intravenous administration of hUC-MSC-ES cells in vivo alleviated oxidative stress damage, reduced inflammation, and reduced apoptosis in the rat MIRI model.

Compared with those of the I/R+Control-ES group, the expressions of TNF-α, IL-1β, and MCP-1 in the I/R+IMTP-ES group were significantly reduced (P<0.01), reactive oxygen species (ROS) was significantly reduced, MDA level was significantly reduced (P all <0.01), and inflammatory cell infiltration was significantly reduced. This proved that the I/R+IMTP-ES group could significantly improve the myocardial oxidative stress damage, inflammation, and apoptosis in the rat I/R model.

### Experimental Example 5

### 1. Experimental Method

A total of 18 Rats (SD rats, male, 8-10 weeks, 250-300 g) were randomly divided into I/R model, Sham group (sham operation group), I/R+PBS group, I/R+Control-ES group, I/R+Blank-ES group, and I/R+IMTP-ES group, and MIRI model was established according to the method above.

After the I/R model was established, the I/R+Control-ES, I/R+Blank-ES, and I/R+IMTP-ES groups were injected once with three types of ES obtained in Example 2 via the tail vein at 400 μg/200 μL. I/R+PBS group: after the I/R model was established, an equal volume of PBS solution (concentration 0.01 M, pH=7.3) was injected into the tail vein. The above four groups repeated the above injections at 3, 5, 14, and 28 d after surgery. The I/R group received no treatment.

Three months after the surgery, the rats were anesthetized by 2% sodium pentobarbital (0.25 mL/100 g) according to part of their body weight. Their heart was exposed by opening the thorax, and the aorta was cannulated through the left ventricle according to the same method, and fixated with 4% paraformaldehyde at 4° C., while other rats were directly sacrificed to collect their heart samples for later detection.

### 2. Test Method

### (1) H&E Staining and Masson Staining Analysis

TABLE 18
Main reagents and manufacturers thereof

| Reagent | Cat. No. | Manufacturer | Locality |
|---|---|---|---|
| Ponceau S | p8330 | Sinopharm | China |
| Acid fuchsin | 71019360 | Sinopharm | China |
| Phosphomolybdic acid | 20029916 | Sinopharm | China |

H&E staining: the H&E staining was conducted according to the above method.

Masson staining: myocardial tissue paraffin sections were routinely dewaxed, and the staining solution was prepared according to the Masson staining kit (Nanjing Senbeijia Biotech). The sections were stained for 10 min, differentiated with acidic ethanol and washed with water, blued with ammonia solution and washed with water, stained with Ponceau fuchsin solution for 10 min, washed with acetic acid, phosphomolybdic acid and acetic acid solution for 1 min each, stained with aniline blue for 2 min, dehydrated with ethanol, treated with xylene for 3 min, and sealed with neutral resin. The fibrosis of each group was observed under a microscope. The infarct size was analyzed using Image J software.

### (2) Echocardiography

Echocardiography was conducted at 4 weeks and 3 months after surgery to detect the left ventricular end-diastolic diameter (LVEDD), left ventricular end-systolic diameter (LVESD), left ventricular fractional shortening (FS), ejection fraction (EF), diastolic interventricular septum thickness (IVSd), diastolic left ventricular posterior wall thickness (LVPWd), and left ventricular mass (LVmass) of the rats in each group.

### (3) Expression Analysis of α-SMA and FITC-Isolectin B4 (BS-I) in Myocardial Tissue

TABLE 19
Main reagents and manufacturers thereof

| Reagent | Cat. No. | Manufacturer | Locality |
|---|---|---|---|
| α-SMA | A17910 | Abclonal | China |
| Cy3-labeled goat anti-rabbit IgG | A27039 | invitrogen | U.S. |
| FITC-Isolectin B4 (BS-I) | L2895 | sigma | U.S. |

Immunofluorescence staining: the cell slides were fixated with 4% paraformaldehyde at 4° C. overnight, rinsed with tris-buffered saline (TBS), incubated with 3% H₂O₂ at room temperature for 20 min, and then washed with TBS and blocked with sheep serum blocking solution for 30 min. After washed with TBS, α-SMA monoclonal antibody was added as the primary antibody, incubated at 4° C. overnight, and washed 3 times with TBS. Cy3-labeled goat anti-rabbit IgG was added as the secondary antibody, incubated at 37° C. for 1 h, and washed 3 times with TBS. The nuclei were stained with DAPI. The slides were sealed with sealing agent, and then observed, scanned, and analyzed under a fluorescence microscope.

### 3. Test Results

### (1) Masson Staining Analysis

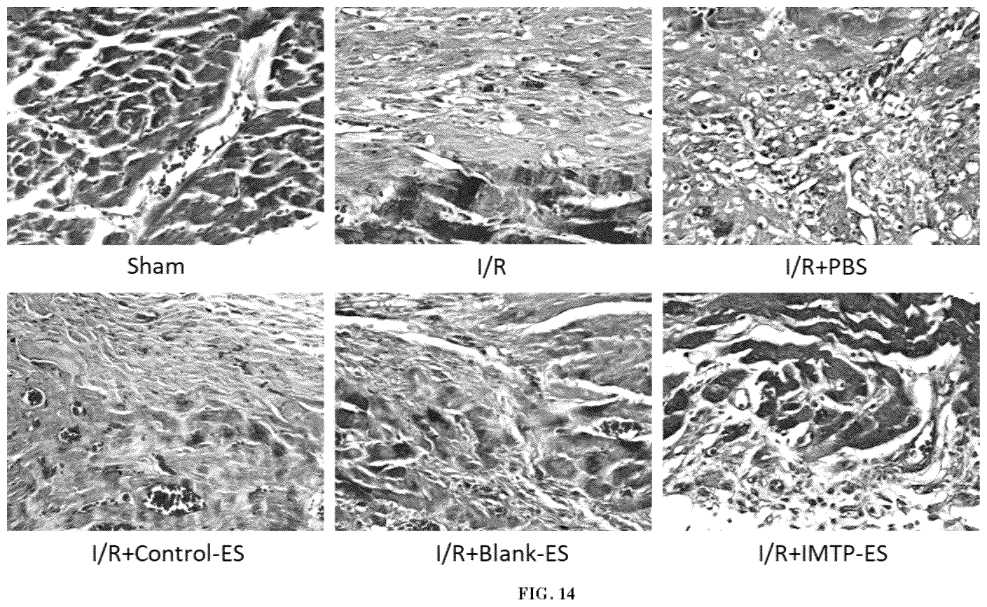

As shown in FIG. 14, the number of CMs in myocardial tissue after I/R injury was significantly reduced, and collagen fibers increased significantly; myocardial fibrosis in the Control-ES and Blank-ES treatment groups was reduced compared with that of the control group and (I/R+PBS) group, and was reduced by 51.9% and 55.3% compared with that of the I/R+PBS group; myocardial fibrosis in the IMTP-ES treatment group was reduced by 83.1% and 81.9% compared with that in the Control-ES and Blank-ES groups (400×, red represented myocardial fibers, blue represented collagen fibers), and the infarct size was also significantly reduced.

### (2) Echocardiography Results

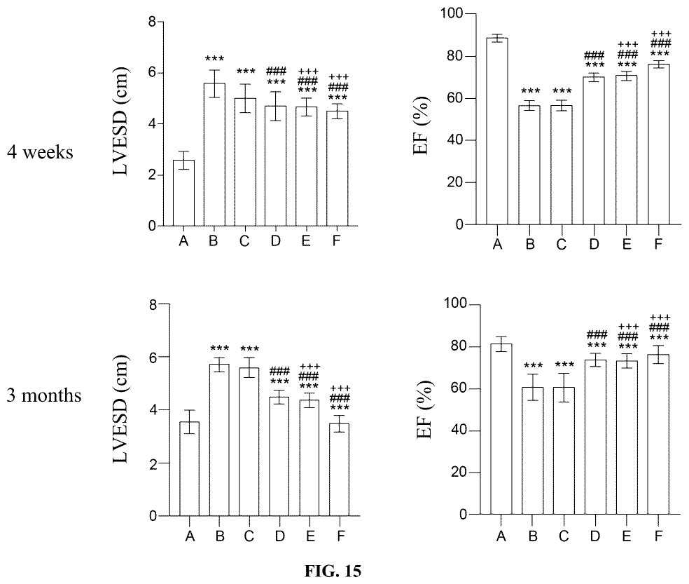

The results are shown in FIG. 15, the ventricular remodeling in the hUC-MSC-ES treatment group was significantly alleviated and the cardiac function was improved.

### (3) Expression of α-SMA and BS-I in Myocardial Tissue

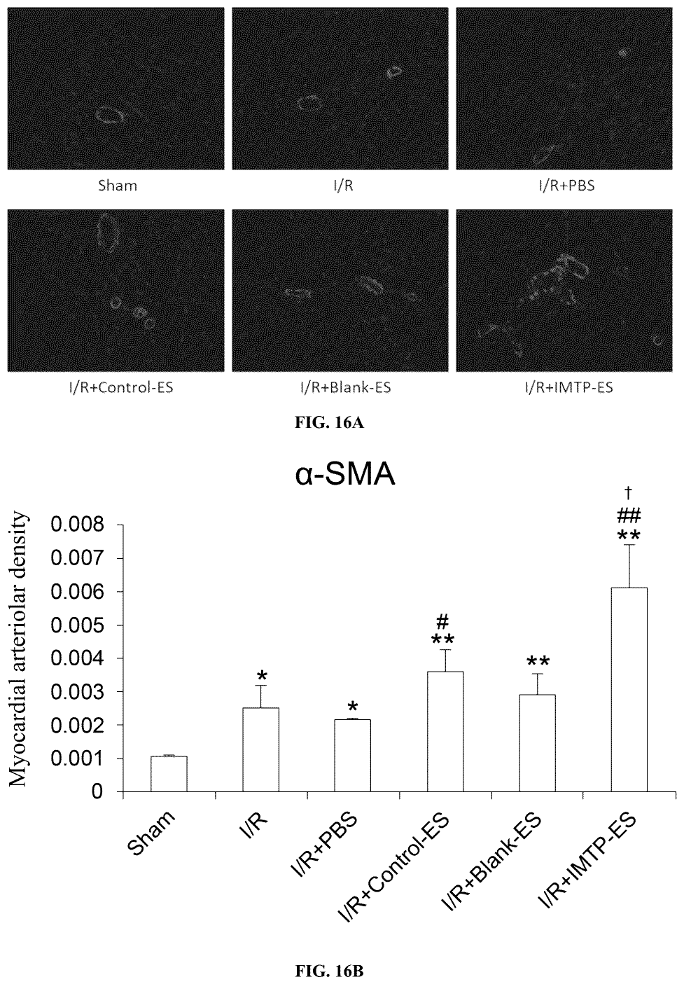

FIG. 16 showed that the expression of α-SMA and FITC-Isolectin B4 (BS-I) in the infarction border zone of MI in the hUC-MSC-ES group increased compared with that in control group. This indicated that the density and diameter of myocardial arterioles per unit area and myocardial capillary density increased, and the arteriole formation and capillary density in the IMTP-ES treatment group increased further than those in the Control-ES and Blank-ES groups.

In summary, ventricular remodeling was significantly reduced and cardiac function was improved in the hUC-MSC-ES treatment group. Immunofluorescence showed that in the infarction border zone of the hUC-MSC-ES group 3 months after surgery, the myocardial capillary density of myocardial arterioles increased. Masson staining showed that the degree of myocardial fibrosis in the treatment group was reduced and the infarct size was reduced. H&E staining showed that no gross tumors and microscopic atypical cell formation were observed in the main organs such as the heart, liver, lung, spleen, whole brain, kidney, large intestine, and quadriceps femoris of rats in the hUC-MSC-ES treatment group. These results suggested that hUC-MSC-ES could treat I/R injury, reduce ventricular remodeling, reduce infarct size, improve cardiac function, promote angiogenesis, reduce myocardial fibrosis and post-MI heart failure, did not increase the risk of tumor formation, and had long-term effectiveness and safety.

### Experimental Example 6 Changes in Dynamic Biological Distribution

### 1. Experimental Method

A total of 36 rats (SD rats, male, 8-10 weeks, 250-300 g) were randomly divided into Control-ES group, I/R+Control-ES, I/R+Blank-ES, and I/R+IMTP-ES group, with 9 rats in each group. Except for the Control-ES group, the MIRI model of rats was established by the aforementioned anterior descending coronary artery ligation method in each group.

After the I/R model was established, the I/R+Control-ES, I/R+Blank-ES, and I/R+IMTP-ES groups were injected once with three types of ES obtained in Example 2 (Control-ES, Blank-ES, IMTP-ES) via the tail vein at 400 μg/200 μL. The Control-ES group consisted of normal rats injected with 400 μg/200 μL of Control-ES. Fluorescence imaging analysis was conducted on each group of rats and major organs 1 h, 24 h, and 48 h after the injection, and the fluorescence intensity of each organ was recorded.

### 2. Test Results

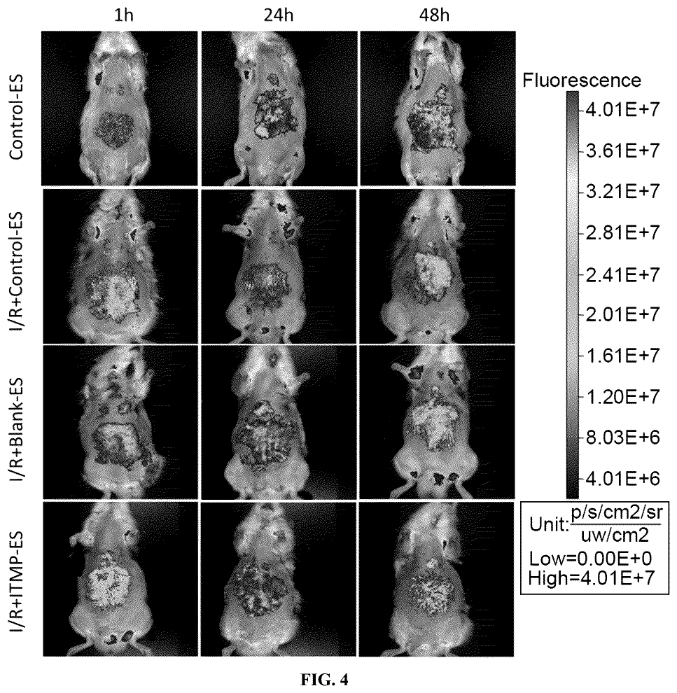

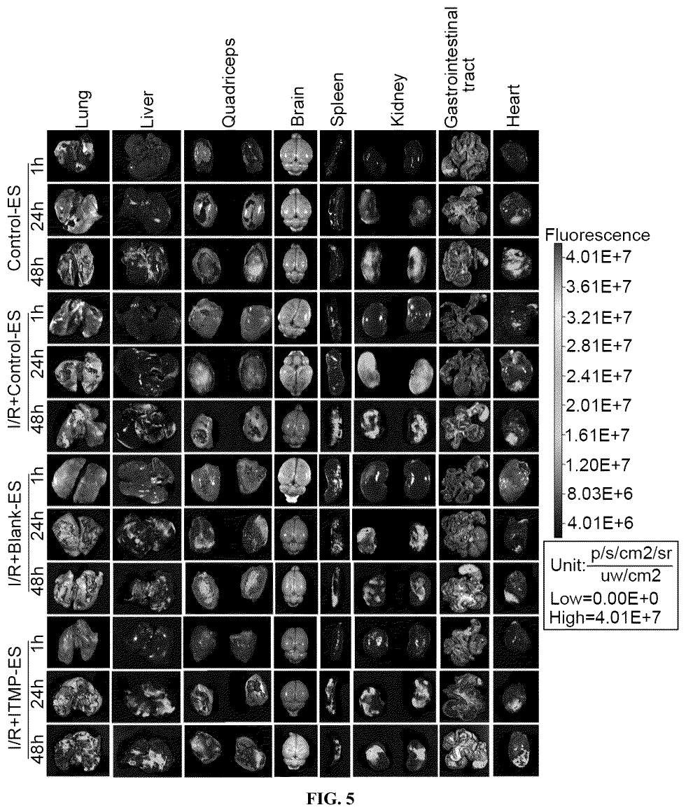

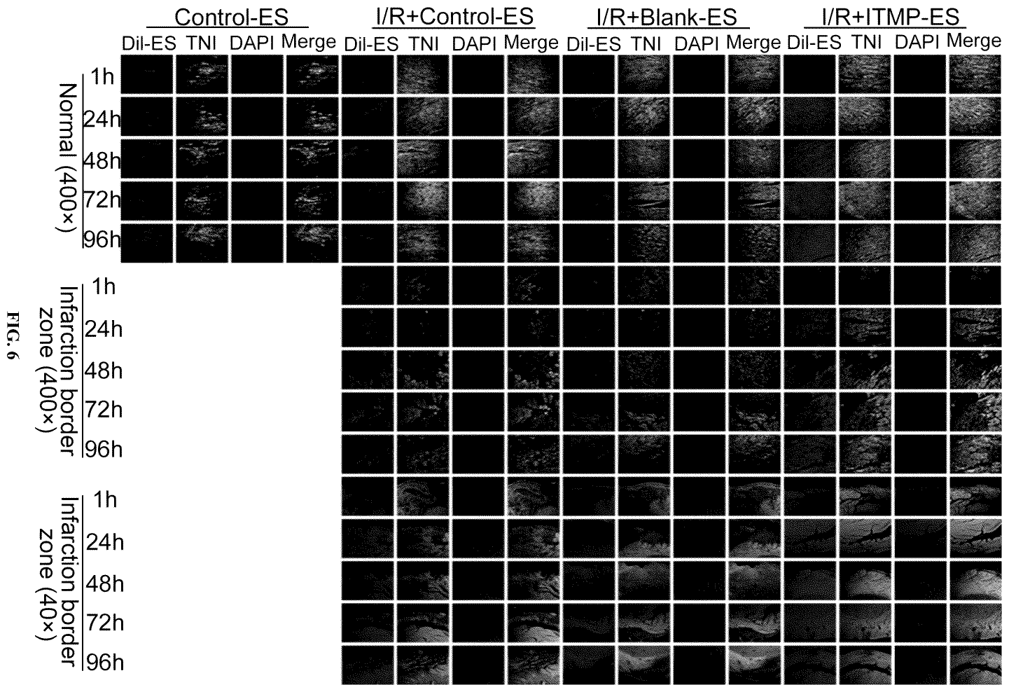

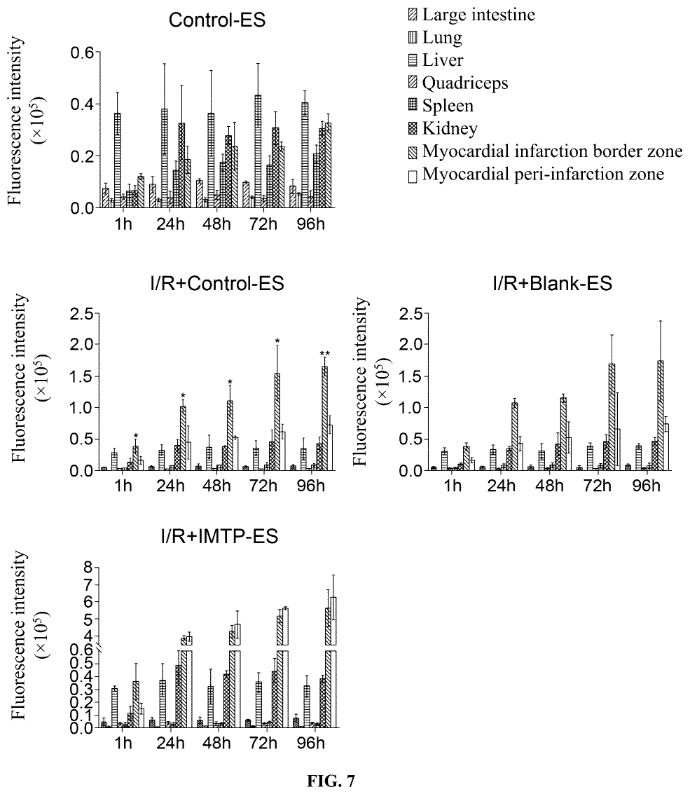

As shown in FIG. 4 to FIG. 7, fluorescence imaging showed that naive hUC-MSC-ES were mainly distributed in muscles, kidneys, liver, and lungs in the first 24 h after entering the body under physiological conditions. In the I/R model, naive hUC-MSC-ES were mainly distributed in the liver, kidneys, spleen, and heart in the first 24 h, and their distribution in the heart increased compared with that in the normal physiological state. The engineered hUC-MSC-ESIMTP-ES targeted to the myocardium by intravenous injection was mainly distributed in the liver, heart, lung, and kidney in the first 24 h, their distribution in the heart was further increased compared with that in the naive hUC-MSC-ES in the I/R model, while its distribution in the quadriceps was significantly reduced. The ES fluorescence in the gastrointestinal tract was less distributed overall. Immunofluorescence showed that the ES fluorescence intensity of the control-ES group was stronger in the liver, kidney, heart, and spleen at each time point, and the fluorescence of other organs was weaker. The ES fluorescence of the I/R+control-ES group and the I/R+Blank-ES group was stronger in the MI infarction border zone (BZ), the non-infarction zone (RZ), the kidney, and the liver. The ES fluorescence of the I/R+IMTP-ES group was stronger in the heart RZ, BZ, kidney, and liver. Under high-power microscope, hUC-MSC-ES in CMs were mainly located in the cytoplasm close to the cell nucleus; part of ES in the liver were located in the hepatic sinusoids, and the other part were phagocytosed by hepatocytes and macrophages; ES fluorescence in the spleen was mainly distributed in the red pulp; ES in the kidney was mainly located in the glomerulus of the renal capsule; ES in the quadriceps femoris were densely distributed in the cytoplasm around the cell nucleus; ES in the intestine were mainly distributed in the epithelium and lamina propria; there was almost no ES fluorescence distribution in the brains of rats in each group at all time points. These results suggested that hUC-MSC-ES were mainly located in the cytoplasm near the nucleus in the body's cells; the liver and spleen might be the early distribution organs of ES, the kidney was the main excretion organ of ES in the early stage, and the gastrointestinal tract was the secondary excretion organ in the later stage. These results showed for the first time the dynamic biological distribution characteristics of naive hUC-MSC-ES and cardiac-targeted hUC-MSC-ES in normal rats and under different conditions of myocardial I/R, providing important kinetic data for the clinical transformation of hUC-MSC-ES in I/R.

### Experimental Example 7 Study on the Therapeutic Mechanism

The transcriptome profile of human hUC-MSC-ES (derived from Control-ES in Example 2) was first analyzed by whole transcriptome microarray (purchased from Cnkingbio Biotechnology). Key mRNAs, miRNAs, LncRNAs, CircRNAs and mutual targeting relationships and important signal pathways in hUC-MSC-ES were discovered. By bioinformatics methods and in combination with corresponding databases, a global signal transduction network, a signal pathway interaction network, and a co-expression regulation network were established. The changes in the signaling pathway interaction network in hUC-MSC-ES were analyzed.

A total of 15 rats (SD rats, male, 8-10 weeks, 250-300 g) were randomly divided into A. Sham group: being only hung with thread but not ligated; B. I/R+PBS group: the I/R model was established by the aforementioned anterior descending branch ligation method, and then 200 μL of PBS with a concentration of 0.01 M and a pH value of 7.3 was injected (n=6); C. I/R+hUC-MSC-ES group: the I/R model was established by the aforementioned anterior descending branch ligation method, and then 400 μg/200 μL hUC-MSC-ES (Control-ES from Example 2) were administered (n=6). 2 h after surgery, cardiac tissues from the infarction border zone of rats with MI were obtained according to the above method.

Bulk transcriptome sequencing was conducted to analyze the signaling pathways and biological molecules that were significantly changed in the myocardial tissue of the infarction border zone of the rat myocardial I/R model after hUC-MSC-ES treatment. The total tSNE cell projection plot of myocardial tissue and changes in proportions of different cellular components in each group of myocardial tissue was analyzed by 10× Genomics single-cell transcriptome sequencing. The biological process, cellular component, and molecular function significantly changed in CMs of rat I/R model after hUC-MSC-ES treatment were analyzed by single cell transcriptome analysis.

Reference: Cell, 2018, 175 (6): 1665-1678. Nat Commun, 2018, 9 (1): 1614. Lancet Oncol, 2018, 19 (3): 382-393.

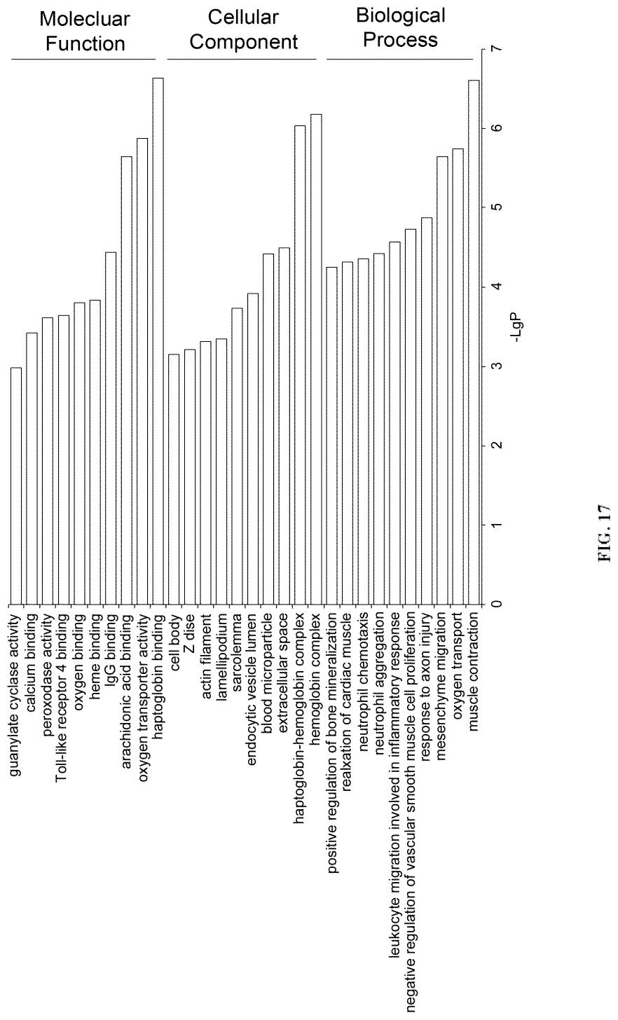

The results showed that compared with those in hUC-MSCs, the biological process, cellular component and molecular function significantly upregulated in hUC-MSC-ES are shown in FIG. 17. In hUC-MSC-ES, the TOP 5 biological processes significantly upregulated included muscle contraction, oxygen transport, mesenchymal migration, response to axonal injury, and negative regulation of vascular smooth muscle cell proliferation. The TOP 5 molecular functions significantly upregulated included haptoglobin binding, oxygen transporter activity, arachidonic acid binding, IgG binding, and heme binding.

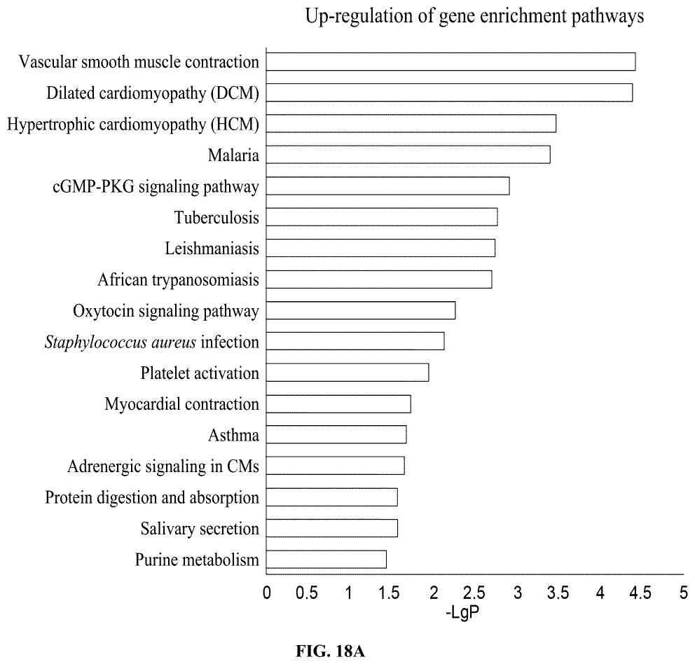

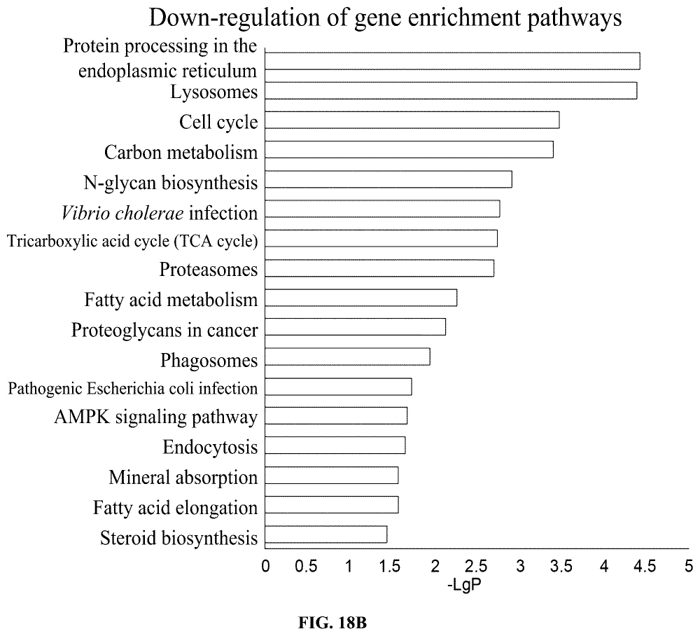

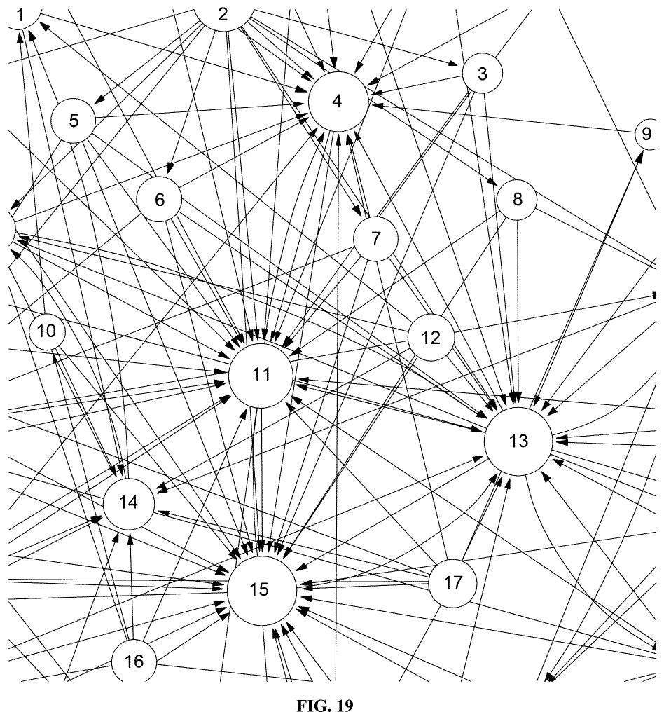

As shown in FIG. 18 and FIG. 19, the cell cycle apoptosis and the apoptosis-related P53 pathway and MAPK pathway were significantly weakened in hUC-MSC-ES.

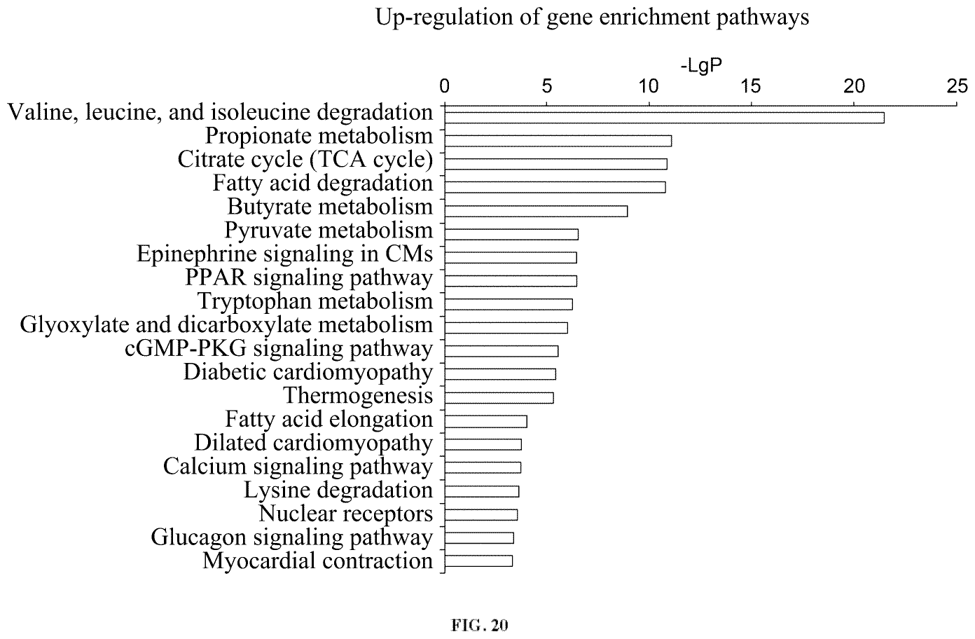

As shown in FIG. 20, in the myocardial tissue of the infarction border zone, the TOP3 signaling pathways significantly upregulated included the degradation of valine, leucine, and isoleucine, propionate metabolism, and citric acid cycle (TCA cycle); the TOP 3 biological processes significantly upregulated included the tricarboxylic acid cycle, positive regulation of macromitochondrial autophagy in response to mitochondrial depolarization, and NADH metabolic process; the TOP 3 molecular functions significantly upregulated included oxidoreductase activity (acting on the CH—CH group of the donor, with flavin as an acceptor), fatty acyl-CoA binding, and NADH dehydrogenase (ubiquinone) activity.

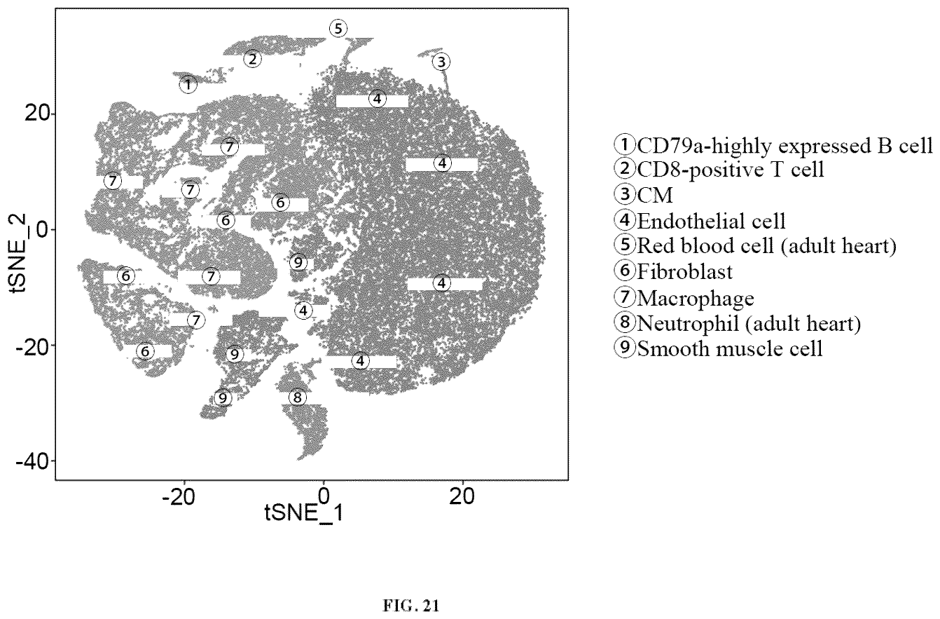

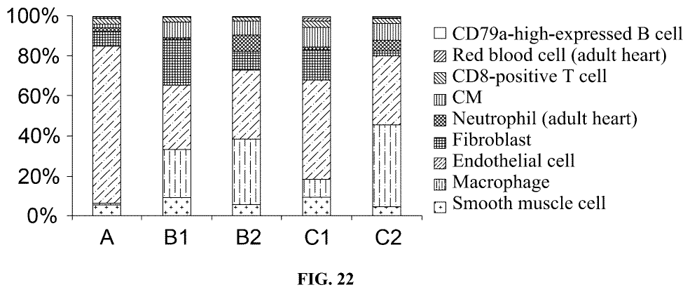

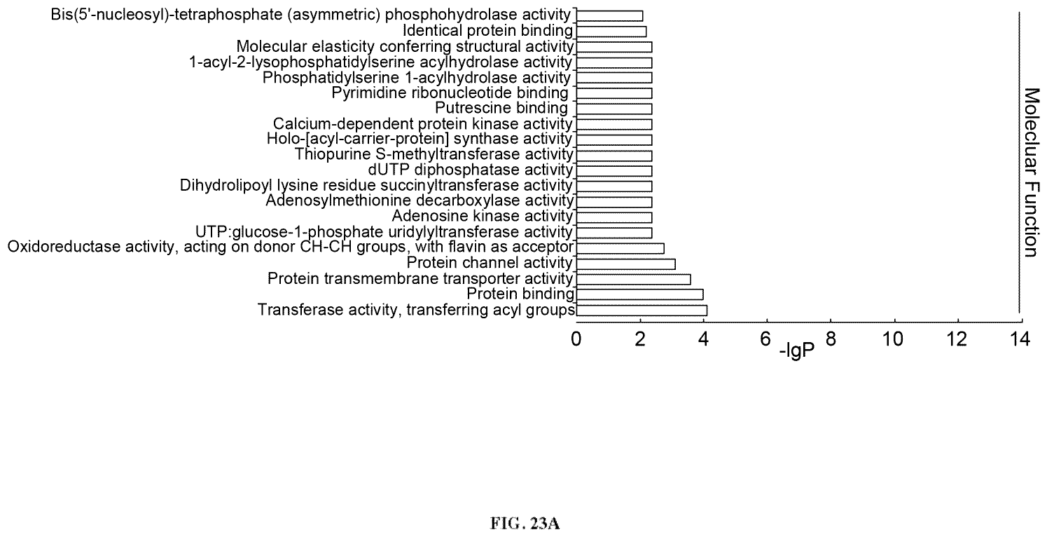

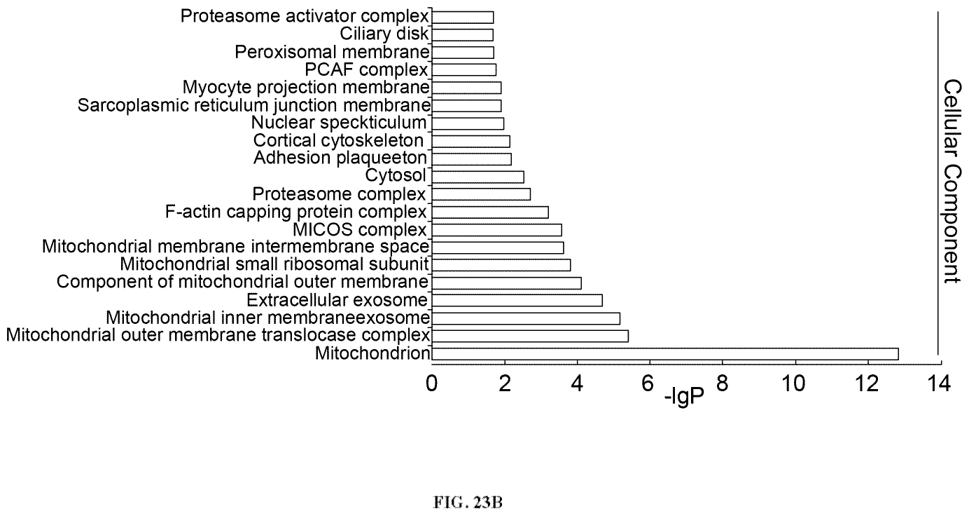

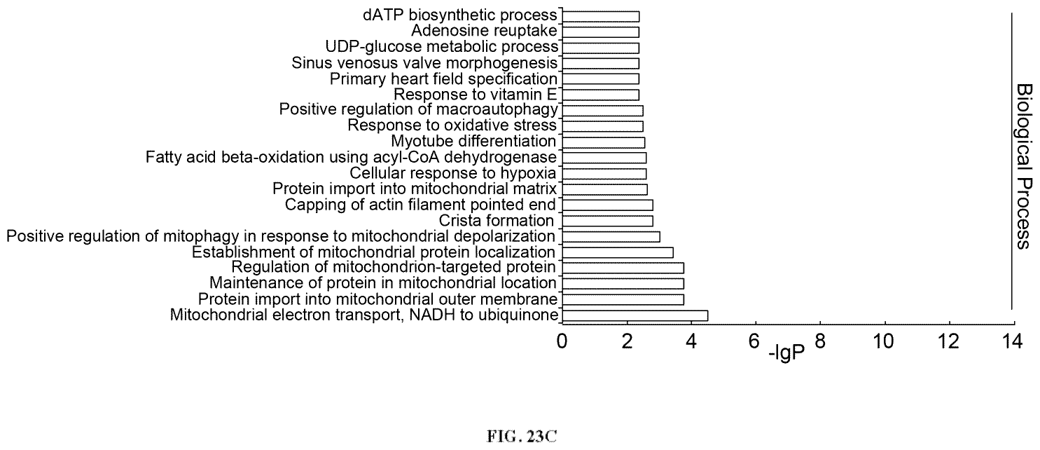

As shown in FIG. 21 to FIG. 23C and the table below, normal myocardial tissue was composed of 9 types of cells and 22 subpopulations, including CMs (1 subpopulation), macrophages (5 subpopulations), endothelial cells (5 subpopulations), fibroblasts (4 subpopulations), smooth muscle cells (3 subpopulations), B cells (1 subpopulation), T cells (1 subpopulation), neutrophils (1 subpopulation), and red blood cells (1 subpopulation). The number of endothelial cells, cardiac fibroblasts (CFbs), and inflammatory cells changed significantly after I/R injury. The most significant change in endothelial cells was a significant decrease after I/R, while CFbs, macrophages, and neutrophils increased significantly, and hUC-MSC-ES treatment alleviated these changes to some extent. In-depth analysis revealed that the expression of genes such as Acadl, IDH, Dlst, and PINK1 in CMs of the hUC-MSC-ES treatment group increased, suggesting that hUC-MSC-ES might reverse the energy metabolism state of CMs after I/R and promote myocardial repair by enhancing fatty acid β-oxidation and improving mitochondrial autophagy. It was also found that after hUC-MSC-ES treatment, the expression of MAPK and p38MAPK in the myocardium was weakened, while the expression of IL1R2 and TLR4 was also reduced, suggesting that hUC-MSC-ES might reduce apoptosis and improve fibrosis through the MAPK pathway, and might reduce inflammatory response through the chemokine pathway, thereby reducing myocardial reperfusion injury and improving prognosis.

TABLE 20

| Cell | A | B1 | B2 | C1 | C2 |
|---|---|---|---|---|---|
| B cell_Cd79a high(Adult-Heart) | 0.29 | 0.51 | 0.33 | 0.73 | 0.60 |
| Cd⁸positive T cell | 2.17 | 2.41 | 1.68 | 2.80 | 2.30 |
| CM | 0.38 | 0.28 | 0.21 | 0.56 | 0.17 |
| Endothelial cell | 81.20 | 39.96 | 41.15 | 59.69 | 42.53 |
| Erythroid cell(Adult-Heart) | 1.08 | 0.20 | 0.42 | 1.70 | 0.56 |
| Fibroblast | 7.25 | 23.00 | 9.29 | 15.40 | 3.08 |
| Macrophage | 0.71 | 23.59 | 33.25 | 9.31 | 41.08 |
| Neutrophil(Adult-Heart) | 1.38 | 0.62 | 8.16 | 0.45 | 4.93 |
| SMC | 5.54 | 9.44 | 5.51 | 9.36 | 4.75 |

B cell_Cd79a high (Adult-Heart): B cell with high expression of Cd79a (adult heart); CM: cardiomyocyte; SMC: smooth muscle cell.

### Experimental Example 8 Storage Stability

The ES (control-ES) prepared in Example 2 were divided into 4 portions, categorized into a PBS group and a preservation solution group, with 2 portions in each group, where the PBS group mixed the ES solution with the PBS solution at a volume ratio of 1:1, and the preservation solution group mixed the ES solution with the colloidal solution prepared in Example 3 at a volume ratio of 1:1. The above groups were stored at −20° C. and −80° C., respectively, and CD63 expression in each group was detected by Western blot at 1 month (−20° C.) and 6 months (−80° C.) (detection method was the same as that of Experimental Example 1). The results showed that the expression of CD63 in the preservation solution group at 1 month increased by 19.5% (p<0.05) compared with that of the PBS group, and at 6 months it increased by 26.7% (p<0.05) compared with that of the PBS group, indicating that the addition of preservation solution made ES more stable.

It is apparent that the above examples are merely listed for clear description, and are not intended to limit the embodiments. The person of ordinary skill in the art may make modifications or variations in other forms based on the above description. There are no need and no way to exhaust all the embodiments. Obvious changes or variations made thereto shall still fall within the protection scope of the present disclosure.

What is claimed is:

1. A method for preparing human umbilical cord mesenchymal stem cell-derived exosomes (hUC-MSC-ES) overexpressing an ischemic myocardium-targeting peptide (IMTP), comprising the following steps: inserting a double-stranded fragment of SEQ ID NO: 1 into a lentiviral vector pCDH-CMV-MCS-EF1-GFP-T2A-puro to obtain a recombinant vector, co-transfecting host cells with the recombinant vector and a packaging system to obtain lentiviral particles, infecting human umbilical cord mesenchymal stem cells (hUC-MSCs) with the lentiviral particles to obtain hUC-MSCs overexpressing the IMTP, preparing a conditioned medium of the hUC-MSCs overexpressing the IMTP, and then collecting the hUC-MSC-ES overexpressing the IMTP from the conditioned medium.

2. The method for preparing hUC-MSC-ES overexpressing an IMTP according to claim 1, wherein the conditioned medium is prepared by the following steps:
suspending subcultured hUC-MSCs overexpressing the IMTP in an exosome-free serum medium to allow culture until a cell confluence reaches 80% to 95%, collecting a supernatant, and then extracting exosomes (ES) from the supernatant.

3. A method for preparing hUC-MSC-ES, comprising the following steps:
suspending subcultured hUC-MSCs in an exsome-free serum medium to allow culture until a cell confluence reaches 80% to 95%, collecting a supernatant, and then extracting exosomes (ES) from the supernatant.

4. The method according to claim 2, wherein extraction of the ES comprises: centrifuging the supernatant at 2,000 g to 3,000 g and 8,000 g to 10,000 g in sequence, filtering an obtained new supernatant, centrifuging an obtained filtrate at 100,000 g to remove a liquid, resuspending a remaining precipitate in a buffer to obtain a solution of hUC-MSC-ES or the hUC-MSC-ES overexpressing the IMTP, and optionally, adding a colloidal solution, to obtain the ES.

5. The method according to claim 4, wherein the colloidal solution is prepared by heating a preservation solution, and the preservation solution comprises 10% weight by volume (w/v) to 30% (w/v) of a plant-derived recombinant human serum albumin, 20 g/L to 40 g/L of trehalose, 30% to 50% of glycerol, 2% to 4% of ectoine, and 0.01 mol/L to 0.02 mol/L of phosphate-buffered saline (PBS); preferably, the colloidal solution and the solution of hUC-MSC-ES or the hUC-MSC-ES overexpressing the IMTP are at a volume ratio of 1-1.5:1-1.5; and preferably, the preservation solution has a pH value of 7.0 to 7.5.

6. HUC-MSC-ES overexpressing the IMTP prepared by the method according to claim 1.

7. A pharmaceutical composition, comprising hUC-MSC-ES or hUC-MSC-ES overexpressing the IMTP prepared by the method according to claim 6 and optionally, a pharmaceutically acceptable auxiliary material.

8. A preservation solution for hUC-MSC-ES, comprising 10% (w/v) to 30% (w/v) of a plant-derived recombinant human serum albumin, 20 g/L to 40 g/L of trehalose, 30% to 50% of glycerol, 2% to 4% of ectoine, and 0.01 mol/L to 0.02 mol/L of PBS.

9. The preservation solution for hUC-MSC-ES according to claim 8, wherein the preservation solution has a pH value of 7.0 to 7.5.

10. A colloidal solution for preserving hUC-MSC-ES, wherein the colloidal solution is prepared by heating the preservation solution according to claim 8; and the heating is conducted at 80° C. to 95° C. for 10 min to 30 min.

11. A method for preserving hUC-MSC-ES, comprising mixing the colloidal solution according to claim 12 with a solution of the hUC-MSC-ES to allow preservation; the colloidal solution and the solution of the hUC-MSC-ES are at a volume ratio of 1-1.5:1-1.5; and the preservation is conducted at not greater than −4° C.

12. A method for preventing, treating, or alleviating MIRI and a related disease thereof, comprising administering to a subject in need thereof hUC-MSC-ES, or the hUC-MSC-ES overexpressing the IMTP prepared by the method according to claim 1.

13. A method for reducing an inflammation and/or CM apoptosis, comprising administering to a subject in need thereof hUC-MSC-ES, the hUC-MSC-ES overexpressing the IMTP prepared by the method according to claim 1.

14. A method for reducing calcium overload and/or promoting angiogenesis, comprising administering to a subject in need thereof hUC-MSC-ES, or the hUC-MSC-ES overexpressing the IMTP prepared by the method according to claim 1.

15. A method for reducing an oxidative stress damage of a CM, comprising administering to a subject in need thereof hUC-MSC-ES, or the hUC-MSC-ES overexpressing the IMTP prepared by the method according to any one of claim 1.

16. A method for reducing infarct size, improving cardiac function, and alleviating myocardial fibrosis, comprising administering to a subject in need thereof hUC-MSC-ES, or the hUC-MSC-ES overexpressing the IMTP prepared by the method according to claim 1.

17. The method according to claim 12, wherein the related disease is one or more selected from the group consisting of inflammation, arrhythmia, ventricular remodeling, myocardial fibrosis, and post-MI heart failure caused by MIRI.

18. The method according to claim 12, wherein the hUC-MSC-ES or the hUC-MSC-ES overexpressing the IMTP are administered at 26 μg/kg to 260 μg/kg.

19. The method according to claim 3, wherein extraction of the ES comprises: centrifuging the supernatant at 2,000 g to 3,000 g and 8,000 g to 10,000 g in sequence, filtering an obtained new supernatant, centrifuging an obtained filtrate at 100,000 g to remove a liquid, resuspending a remaining precipitate in a buffer to obtain a solution of hUC-MSC-ES or the hUC-MSC-ES overexpressing the IMTP, and optionally, adding a colloidal solution, to obtain the ES.

20. HUC-MSC-ES prepared according to claim 3.


SEQUENCE LISTING (4 sequences)

SEQ ID NO: 1 - DNA, 1281 bp, synthetic construct
  source 1..1281: mol_type = other DNA; note = IMTP+LAMP2B insertion sequence
gccaccatggtgtgcttccgcctcttcccggttccgggctcagggctcgttctggtctgcctagtcctgggagctgtgcggtcttatgcagctcgatgtagcacttcaatgctgaaagcatgttccggaggtttggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagcccaagagtgttcgctggatgatgacaccattctaatcccaattatagttggtgctggtctttcaggcttgattatcgttatagtgattgcttacgtaattggcagaagaaaaagttatgctggatatcagactctgtaa

SEQ ID NO: 2 - DNA, 1239 bp, synthetic construct
  source 1..1239: mol_type = other DNA; note = CDS region of LAMP2B
gccaccatggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagcccaagagtgttcgctggatgatgacaccattctaatcccaattatagttggtgctggtctttcaggcttgattatcgttatagtgattgcttacgtaattggcagaagaaaaagttatgctggatatcagactctgtaa

SEQ ID NO: 3 - DNA, 27 bp, synthetic construct
  source 1..27: mol_type = other DNA; note = base sequence encoding IMTP
tgtagcacttcaatgctgaaagcatgt

SEQ ID NO: 4 - protein, 9 aa, synthetic construct
  source 1..9: note = IMTP
CSTSMLKAC